(19) United States
(12) Patent Application Publication
(10) Pub. No.: US 2024/0417399 A1
(43) Pub. Date: Dec. 19, 2024

(54) ARYL HYDROCARBON RECEPTOR (AHR) MODULATORS AND THERAPEUTIC USES THEREOF

(71) Applicant: GALILEO BIOSYSTEMS, INC., SAN DIEGO, CA (US)

(72) Inventors: Michael Howell, San Diego, CA (US); Steven Bruce Cohen, San Diego, CA (US); Elizabeth Paige Stout, San Diego, CA (US); Venkata Rami Reddy Macherla, San Diego, CA (US); Phil S. Baran, San Diego, CA (US); Eduardo Esquenazi, La Jolla, CA (US); Tamara Schwent, Sarasota, FL (US); Farhana Barmare, San Diego, CA (US)

(21) Appl. No.: 18/700,236

(22) PCT Filed: Oct. 13, 2022
(86) PCT No.: PCT/US2022/046595

§ 371 Date: Apr. 10, 2024

Related U.S. Application Data
(60) Provisional application No. 63/262,514, filed on Oct. 14, 2021.

(51) Int. Cl. C07D 471/04; A61K 31/4184; A61K 31/437; A61P 17/06; A61P 29/00; C07D 403/06

(52) U.S. Cl. CPC C07D 471/04; A61K 31/4184; A61K 31/437; A61P 17/06; A61P 29/00; C07D 403/06

(57) ABSTRACT

Disclosed herein are small molecule aryl hydrocarbon receptor (AhR) modulator compositions, pharmaceutical compositions, the use and preparation thereof.

### BACKGROUND

### Field of the Invention

The present invention relates to the fields of chemistry and medicine. More particularly, the present invention relates to substituted benzimidazolyl pyrrolopyridine compounds as small molecule aryl hydrocarbon receptor (AhR) modulators, compositions, their preparation, and their use as therapeutic agents.

### Description of the Related Art

Aryl hydrocarbon receptor (AhR) is a ligand-dependent transcriptional factor present in the cytoplasm as an inactive complex that translocates into the nucleus upon ligand binding. AhR is released in the nucleus and heterodimerizes with AhR nuclear translocator to bind specific genomic regions and induce transcription of target genes to produce a range of gene expression biomarkers including CYP1A1 and AhR repressor. AhR signaling is involved in several critical pathways including sensing and regulating the immune response, regulation of T cell differentiation and T cell mediated immune responses, suppression of inflammation, and development of autoimmune disease.

AhR is expressed in immune cells and regulates innate and adaptive immune responses which impact the balance of diseases mediated by Th1 activity, Th2 activity, Th17 activity, and T regulatory responses. AhR is also widely expressed in epithelial, endothelial and stromal cells in the skin and activation of AhR reduces skin inflammation and IL-17 mediated inflammation.

Ligands of AhR can be small molecule organic compounds that are naturally occurring or produced synthetically. Environmental polycyclic aromatic hydrocarbons and dioxins bind to AhR to induce reactive oxygen species (ROS). Increased ROS levels trigger cytokine production leading to barrier disruption and chronic skin inflammation.

Currently there is a need to provide new and alternative treatment therapies for diseases mediated by aryl hydrocarbon receptor (AhR).

### SUMMARY

Disclosed herein are compound having the structure of the formula (I):

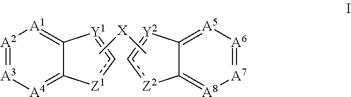

or a pharmaceutically acceptable salt thereof, wherein:
A¹, A², A³, A⁴, A⁵, A⁶, A⁷, and A⁸ are each independently C(R¹) or N, wherein at least one of A¹, A², A³, A⁴, A⁵, A⁶, A⁷, and A⁸ is C(R⁶) or N;
Y¹, Y², Z¹, and Z² are each independently selected from the group consisting of O, S, C(R²), and NR³, wherein at least one of Y¹, Y², Z¹, and Z² is NR³;
X is selected from the group consisting of (C(R⁴)₂)ₘ, (C═NR⁵), (C═O), (C═S), O, S, and SO₂;
each R¹ is independently selected from the group consisting of hydrogen, halo, hydroxy, optionally substituted C₁₋₈ alkyl, optionally substituted C₁₋₈ haloalkyl, optionally substituted C₁₋₈ alkoxy, optionally substituted C₆₋₁₀ aryloxy, optionally substituted C₁₋₈ alkyl-O-sulfonyl, optionally substituted C₁₋₈ haloalkyl-O-sulfonyl, optionally substituted C₃₋₁₀ carbocyclyl, optionally substituted C₄₋₁₀ carbocyclyl(C₁-C₆)alkyl, optionally substituted 3-10 membered heterocyclyl, optionally substituted 3-10 membered heterocyclyl(C₁-C₆)alkyl, optionally substituted C₆₋₁₀ aryl, optionally substituted C₆₋₁₀ aryl(C₁-C₆)alkyl, optionally substituted 5-10 membered heteroaryl, and optionally substituted 5-10 membered heteroaryl(C₁-C₆)alkyl, or independently two R¹ groups can be taken together with the atoms to which they are attached to form a ring selected from the group consisting of optionally substituted 5-10 membered heterocyclic, optionally substituted 5-10 membered heteroaryl, optionally substituted C₆₋₁₀ aryl, and optionally substituted C₄₋₁₀ carbocyclyl;
each R² and R³ is independently selected from the group consisting of hydrogen, halo, hydroxy, and optionally substituted C₁₋₈ alkyl or is absent;
each R⁴ is each independently selected from the group consisting of hydrogen, halo, hydroxy, and optionally substituted C₁₋₈ alkyl;
R⁵ is hydrogen or C₁₋₈ alkyl;
each R⁶ is independently selected from the group consisting of hydroxy, bromo, chloro, optionally substituted C₁₋₈ haloalkyl, optionally substituted C₆₋₁₀ aryloxy, optionally substituted C₁₋₈ haloalkyl-O-sulfonyl, optionally substituted C₃₋₁₀ carbocyclyl, optionally substituted C₄₋₁₀ carbocyclyl(C₁-C₆)alkyl, optionally substituted 3-10 membered heterocyclyl, optionally substituted 3-10 membered heterocyclyl(C₁-C₆)alkyl, optionally substituted C₆₋₁₀ aryl, optionally substituted C₆₋₁₀ aryl(C₁-C₆)alkyl, optionally substituted 5-10 membered heteroaryl, and optionally substituted 5-10 membered heteroaryl(C₁-C₆)alkyl, or independently two R⁶ groups can be taken together with the atoms to which they are attached to form a ring selected from the group consisting of optionally substituted 5-10 membered heterocyclic, optionally substituted 5-10 membered heteroaryl, optionally substituted C₆₋₁₀ aryl, and optionally substituted C₄₋₁₀ carbocyclyl;
m is an integer from 1 to 6; and
each [symbol] is independently single bond or double bond, wherein adjacent [symbol] are not both double bonds.

Other embodiments disclosed herein include a pharmaceutical composition comprising a therapeutically effective amount of a compound disclosed herein and a pharmaceutically acceptable excipient.

In some embodiments, the compounds disclosed herein are broadly effective in treating a host of diseases mediated by aryl hydrocarbon receptor, and specifically including those associated with Th2 activity and Th17 activity. Accordingly, compounds disclosed herein are active therapeutics for a diverse set of diseases or disorders that include or that produces a symptom which include, but are not limited to: a dermatologic disease selected from atopic dermatitis, eczema, psoriasis, contact dermatitis, vitiligo, hidradenitis suppurativa, lichen planus, ichthyosis, palmoplantar pustulosis, dermatomyositis, netherton's syndrome, fibrosis, scleroderma, basal cell carcinoma, squamous cell carcinoma, merkel cell carcinoma, skin aging, bullous pemphigoid, sarcoidosis, chronic urticaria, rosacea, alopecia areata, pyoderma gangrenosum, cutaneous T cell lymphoma; respiratory disease selected from asthma, chronic obstructive pulmonary disorder, idiopathic pulmonary fibrosis, bronchitis, cystic fibrosis, acute respiratory distress syndrome, interstitial lung disease; an oncologic disease selected from melanoma, non-small cell lung cancer, prostate cancer, pancreatic cancer, bladder cancer, mesothelioma; an autoimmune disease selected from ankylosing spondylitis, rheumatoid arthritis, psoriatic arthritis, systemic lupus erythematosus, diabetic nephropathy, Graves' disease, multiple sclerosis, type 1 diabetes, Sjogren's disease, neuromyelitis optica, amyloidosis, IgG4 disease, Behcet's disease, Celiac disease, fibromyalgia, ulcerative colitis, Crohn's disease, uveitis; and an immunologic disease selected from COVID-19, SARS-COV2, graft versus host disease, cytokine release syndrome, STAT3 gain of function, interferonopathies, Kawasaki disease, multi-inflammatory syndrome.

In some embodiments, the compounds disclosed herein are used to treat diseases or conditions or that produces a symptom in a subject which include, but not limited to: a dermatologic disease selected from atopic dermatitis, eczema, psoriasis, contact dermatitis, vitiligo, hidradenitis suppurativa, lichen planus, ichthyosis, palmoplantar pustulosis, dermatomyositis, netherton's syndrome, fibrosis, scleroderma, basal cell carcinoma, squamous cell carcinoma, merkel cell carcinoma, skin aging, bullous pemphigoid, sarcoidosis, chronic urticaria, rosacea, alopecia areata, pyoderma gangrenosum, cutaneous T cell lymphoma; respiratory disease selected from asthma, chronic obstructive pulmonary disorder, idiopathic pulmonary fibrosis, bronchitis, cystic fibrosis, acute respiratory distress syndrome, interstitial lung disease; an oncologic disease selected from melanoma, non-small cell lung cancer, prostate cancer, pancreatic cancer, bladder cancer, mesothelioma; an autoimmune disease selected from ankylosing spondylitis, rheumatoid arthritis, psoriatic arthritis, systemic lupus erythematosus, diabetic nephropathy, Graves' disease, multiple sclerosis, type 1 diabetes, Sjogren's disease, neuromyelitis optica, amyloidosis, IgG4 disease, Behcet's disease, Celiac disease, fibromyalgia, ulcerative colitis, Crohn's disease, uveitis; and an immunologic disease selected from COVID-19, SARS-CoV2, graft versus host disease, cytokine release syndrome, STAT3 gain of function, interferonopathies, Kawasaki disease, multi-inflammatory syndrome.

### BRIEF DESCRIPTION OF THE DRAWINGS

FIG. 1A illustrates the effect of the compounds of the present disclosure on production of CYP1A1.

FIG. 1B illustrates the effect of the compounds of the present disclosure on production of CCL20.

FIG. 1C illustrates the effect of the compounds of the present disclosure on production of IL-17A.

FIG. 2A illustrates the effect of the compounds of the present disclosure on ear thickness.

FIG. 2B illustrates the effect of the compounds of the present disclosure on production of IL-17A in the ear.

FIG. 3 illustrates the effect of the compounds of the present disclosure on body weight.

FIG. 4A illustrates the effect of the compounds of the present disclosure on production of HBD4 in tissue.

FIG. 4B illustrates the effect of the compounds of the present disclosure on production of S100A7 in tissue.

FIG. 4C illustrates the effect of the compounds of the present disclosure on production of Elafin in tissue.

FIG. 4D illustrates the effect of the compounds of the present disclosure on production of CCL20 in tissue.

FIG. 5A depicts concentration response curves for CCL2 release and cell viability of human keratinocytes exposed to Compound 10.

FIG. 5B depicts concentration response curves for CCL2 release and cell viability of human keratinocytes exposed to tapiranof.

FIG. 6A depicts concentration response curves for IL-8 release and cell viability of human keratinocytes exposed to Compound 10.

FIG. 6B depicts concentration response curves for IL-8 release and cell viability of human keratinocytes exposed to tapiranof.

FIG. 7A depicts concentration response curves for IL-2 and IL-4 release and cell viability of T-cells exposed to Compound 10.

FIG. 7B depicts concentration response curves for IL-2 and IL-4 release and cell viability of T-cells exposed to tapiranof.

### DETAILED DESCRIPTION

In some embodiments, provided herein are substituted benzimidazolyl pyrrolopyridine compounds that function as aryl hydrocarbon receptor modulators. Various embodiments of these compounds include compounds having the structure of Formula (I) as described above or pharmaceutically acceptable salts thereof. The structure of Formula (I) encompasses all stereoisomers and racemic mixtures, including the following structures and mixtures thereof:

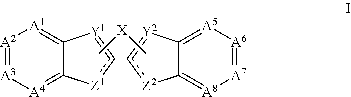

In some embodiments of compounds of Formula (I):
A¹, A², A³, A⁴, A⁵, A⁶, A⁷, and A⁸ are each independently C(R¹) or N, wherein at least one of A¹, A², A³, A⁴, A⁵, A⁶, A⁷, and A⁸ is C(R⁶) or N;
Y¹, Y², Z¹, and Z² are each independently selected from the group consisting of O, S, C(R²), and NR³, wherein at least one of Y¹, Y², Z¹, and Z² is NR³;
X is selected from the group consisting of (C(R⁴)₂)ₘ, (C═NR⁵), (C═O), (C═S), O, S, and SO₂;
each R¹ is independently selected from the group consisting of hydrogen, halo, hydroxy, optionally substituted C₁₋₈ alkyl, optionally substituted C₁₋₈ haloalkyl, optionally substituted C₁₋₈ alkoxy, optionally substituted C₆₋₁₀ aryloxy, optionally substituted C₁₋₈ alkyl-O-sulfonyl, optionally substituted C₁₋₈ haloalkyl-O-sulfonyl, optionally substituted C₃₋₁₀ carbocyclyl, optionally substituted C₄₋₁₀ carbocyclyl(C₁-C₆)alkyl, optionally substituted 3-10 membered heterocyclyl, optionally substituted 3-10 membered heterocyclyl(C₁-C₆)alkyl, optionally substituted C₆₋₁₀ aryl, optionally substituted C₆₋₁₀ aryl(C₁-C₆)alkyl, optionally substituted 5-10 membered heteroaryl, and optionally substituted 5-10 membered heteroaryl(C₁-C₆)alkyl, or independently two R¹ groups can be taken together with the atoms to which they are attached to form a ring selected from the group consisting of optionally substituted 5-10 membered heterocyclic, optionally substituted 5-10 membered heteroaryl, optionally substituted C₆₋₁₀ aryl, and optionally substituted C₄₋₁₀ carbocyclyl;
each R² and R³ is independently selected from the group consisting of hydrogen, halo, hydroxy, and optionally substituted C₁₋₈ alkyl or is absent;
each R⁴ is each independently selected from the group consisting of hydrogen, halo, hydroxy, and optionally substituted C₁₋₈ alkyl;
R⁵ is hydrogen or C₁₋₈ alkyl;
each R⁶ is independently selected from the group consisting of hydroxy, bromo, chloro, optionally substituted C₁₋₈ haloalkyl, optionally substituted C₆₋₁₀ aryloxy, optionally substituted C₁₋₈ haloalkyl-O-sulfonyl, optionally substituted C₃₋₁₀ carbocyclyl, optionally substituted C₄₋₁₀ carbocyclyl(C₁-C₆)alkyl, optionally substituted 3-10 membered heterocyclyl, optionally substituted 3-10 membered heterocyclyl(C₁-C₆)alkyl, optionally substituted C₆₋₁₀ aryl, optionally substituted C₆₋₁₀ aryl(C₁-C₆)alkyl, optionally substituted 5-10 membered heteroaryl, and optionally substituted 5-10 membered heteroaryl(C₁-C₆)alkyl, or independently two R⁶ groups can be taken together with the atoms to which they are attached to form a ring selected from the group consisting of optionally substituted 5-10 membered heterocyclic, optionally substituted 5-10 membered heteroaryl, optionally substituted C₆₋₁₀ aryl, and optionally substituted C₄₋₁₀ carbocyclyl;
m is an integer from 1 to 6; and
each [symbol] is independently single bond or double bond, wherein adjacent [symbol] are not both double bonds.

Several embodiments of the compounds include compounds having the structure of Formula (II) or pharmaceutically acceptable salts thereof. The structure of Formula (II) encompasses all stereoisomers and racemic mixtures, including the following structures and mixtures thereof.

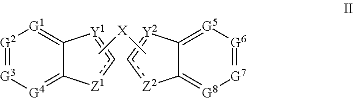

In some embodiments of compounds of Formula (II):
G¹, G², G³, G⁴, G⁵, G⁶, G⁷, and G⁸ are each independently C(R¹) or N;
Y¹, Y², Z¹, and Z² are each independently selected from the group consisting of O, S, C(R²), and NR³, wherein at least one of Y¹, Y², Z¹, and Z² is NR³;
X is selected from the group consisting of (C(R⁴)₂)ₘ, (C═NR⁵), (C═O), (C═S), O, S, and SO₂;
each R¹ is independently selected from the group consisting of hydrogen, halo, hydroxy, optionally substituted C₁₋₈ alkyl, optionally substituted C₁₋₈ haloalkyl, optionally substituted C₁₋₈ alkoxy, optionally substituted C₆₋₁₀ aryloxy, optionally substituted C₁₋₈alkyl-O-sulfonyl, optionally substituted C₁₋₈ haloalkyl-O-sulfonyl, optionally substituted C₃₋₁₀ carbocyclyl, optionally substituted C₄₋₁₀ carbocyclyl(C₁-C₆)alkyl, optionally substituted 3-10 membered heterocyclyl, optionally substituted 3-10 membered heterocyclyl(C₁-C₆)alkyl, optionally substituted C₆₋₁₀ aryl, optionally substituted C₆₋₁₀ aryl(C₁-C₆)alkyl, optionally substituted 5-10 membered heteroaryl, and optionally substituted 5-10 membered heteroaryl(C₁-C₆)alkyl, or independently two R¹ groups can be taken together with the atoms to which they are attached to form a ring selected from the group consisting of optionally substituted 5-10 membered heterocyclic, optionally substituted 5-10 membered heteroaryl, optionally substituted C₆₋₁₀ aryl, and optionally substituted C₄₋₁₀ carbocyclyl;
each R² and R³ is independently selected from the group consisting of hydrogen, halo, hydroxy, and optionally substituted C₁₋₈ alkyl or is absent;
each R⁴ is each independently selected from the group consisting of hydrogen, halo, hydroxy, and optionally substituted C₁₋₈ alkyl;
R⁵ is hydrogen or C₁₋₈alkyl;
m is an integer from 1 to 6; and
each [symbol] is independently single bond or double bond, wherein adjacent [symbol] are not both double bonds.

In some embodiments of compounds of Formula (I) or their pharmaceutically acceptable salts, A¹ is N. In some embodiments of compounds of Formula (I) or their pharmaceutically acceptable salts, A² is N. In some embodiments of compounds of Formula (I) or their pharmaceutically acceptable salts, A³ is N. In some embodiments of compounds of Formula (I) or their pharmaceutically acceptable salts, A⁴ is N. In some embodiments of compounds of Formula (I) or their pharmaceutically acceptable salts, A⁵ is N. In some embodiments of compounds of Formula (I) or their pharmaceutically acceptable salts, A⁶ is N. In some embodiments of compounds of Formula (I) or their pharmaceutically acceptable salts, A⁷ is N. In some embodiments of compounds of Formula (I) or their pharmaceutically acceptable salts, A⁸ is N.

In some embodiments of compounds of Formula (I) or their pharmaceutically acceptable salts, at least two of A¹, A², A³, A⁴, A⁵, A⁶, A⁷, and A⁸ are N. In some embodiments of compounds of Formula (I) or their pharmaceutically acceptable salts, at least three of A¹, A², A³, A⁴, A⁵, A⁶, A⁷, and A⁸ are N. In some embodiments of compounds of Formula (I) or their pharmaceutically acceptable salts, at least four of A¹, A², A³, A⁴, A⁵, A⁶, A⁷, and A⁸ are N.

In some embodiments of compounds of Formula (I) or their pharmaceutically acceptable salts, A′ is C(R¹). In some embodiments of compounds of Formula (I) or their pharmaceutically acceptable salts, A² is C(R¹). In some embodiments of compounds of Formula (I) or their pharmaceutically acceptable salts, A³ is C(R¹). In some embodiments of compounds of Formula (I) or their pharmaceutically acceptable salts, A⁴ is C(R¹). In some embodiments of compounds of Formula (I) or their pharmaceutically acceptable salts, A⁵ is C(R¹). In some embodiments of compounds of Formula (I) or their pharmaceutically acceptable salts, A⁶ is C(R¹). In some embodiments of compounds of Formula (I) or their pharmaceutically acceptable salts, A⁷ is C(R¹). In some embodiments of compounds of Formula (I) or their pharmaceutically acceptable salts, A⁸ is C(R¹).

In some embodiments of compounds of Formula (I) or their pharmaceutically acceptable salts, A¹ is C(R⁶). In some embodiments of compounds of Formula (I) or their pharmaceutically acceptable salts, A² is C(R⁶). In some embodiments of compounds of Formula (I) or their pharmaceutically acceptable salts; A³ is C(R⁶). In some embodiments of compounds of Formula (I) or their pharmaceutically acceptable salts; A⁴ is C(R⁶). In some embodiments of compounds of Formula (I) or their pharmaceutically acceptable salts, A⁵ is C(R⁶). In some embodiments of compounds of Formula (I) or their pharmaceutically acceptable salts, A⁶ is C(R⁶). In some embodiments of compounds of Formula (I) or their pharmaceutically acceptable salts, A⁷ is C(R⁶). In some embodiments of compounds of Formula (I) or their pharmaceutically acceptable salts, A⁸ is C(R⁶).

In some embodiments of compounds of Formula (II) or their pharmaceutically acceptable salts, G¹ is N. In some embodiments of compounds of Formula (II) or their pharmaceutically acceptable salts, G² is N. In some embodiments of compounds of Formula (II) or their pharmaceutically acceptable salts, G³ is N. In some embodiments of compounds of Formula (II) or their pharmaceutically acceptable salts, G⁴ is N. In some embodiments of compounds of Formula (II) or their pharmaceutically acceptable salts, G⁵ is N. In some embodiments of compounds of Formula (II) or their pharmaceutically acceptable salts, G⁶ is N. In some embodiments of compounds of Formula (I)I or their pharmaceutically acceptable salts, G⁷ is N. In some embodiments of compounds of Formula (II) or their pharmaceutically acceptable salts, G⁸ is N.

In some embodiments of compounds of Formula (II) or their pharmaceutically acceptable salts, at least two of G′, G², G³, G⁴, G⁵, G⁶, G⁷, and G⁸ are N. In some embodiments of compounds of Formula (II) or their pharmaceutically acceptable salts, at least three of G¹, G², G³, G⁴, G⁵, G⁶, G⁷, and G⁸ are N. In some embodiments of compounds of Formula (II) or their pharmaceutically acceptable salts, at least four of G¹, G², G³, G⁴, G⁵, G⁶, G⁷, and G⁸ are N.

In some embodiments of compounds of Formula (II) or their pharmaceutically acceptable salts, G¹ is C(R¹). In some embodiments of compounds of Formula (II) or their pharmaceutically acceptable salts, G² is C(R¹). In some embodiments of compounds of Formula (II) or their pharmaceutically acceptable salts, G³ is C(R¹). In some embodiments of compounds of Formula (II) or their pharmaceutically acceptable salts, G⁴ is C(R¹). In some embodiments of compounds of Formula (II) or their pharmaceutically acceptable salts, G⁵ is C(R¹). In some embodiments of compounds of Formula (II) or their pharmaceutically acceptable salts, G⁶ is C(R¹). In some embodiments of compounds of Formula (II) or their pharmaceutically acceptable salts, G⁷ is C(R¹). In some embodiments of compounds of Formula (II) or their pharmaceutically acceptable salts, G⁸ is C(R¹).

In some embodiments of compounds of Formula (I), Formula (II), or their pharmaceutically acceptable salts, at least one R¹ is hydrogen. In some embodiments of compounds of Formula (I), Formula (II), or their pharmaceutically acceptable salts, at least one R¹ is halo. In some embodiments of compounds of Formula (I), Formula (II), or their pharmaceutically acceptable salts, at least one R¹ is F. In some embodiments of compounds of Formula (I), Formula (II), or their pharmaceutically acceptable salts, at least one R¹ is Cl. In some embodiments of compounds of Formula (I), Formula (II), or their pharmaceutically acceptable salts, at least one R¹ is Br. In some embodiments of compounds of Formula (I) or their pharmaceutically acceptable salts; at least one R¹ is C₁₋₈ alkyl. In some embodiments of compounds of Formula (I), Formula (II), or their pharmaceutically acceptable salts, at least one R¹ is methyl. In some embodiments of compounds of Formula (I), Formula (II), or their pharmaceutically acceptable salts, at least one R¹ is C₁₋₈ haloalkyl. In some embodiments of compounds of Formula (I), Formula (II), or their pharmaceutically acceptable salts, at least one R¹ is CF₃.

In some embodiments of compounds of Formula (I) or its pharmaceutically acceptable salts, at least one R⁶ is C₁₋₈ haloalkyl. In some embodiments of compounds of Formula (I) or its pharmaceutically acceptable salts, at least one R⁶ is CF₃.

In some embodiments of compounds of Formula (I), Formula (II), or their pharmaceutically acceptable salts, Y¹ is C(R²).

In some embodiments of compounds of Formula (I), Formula (II), or their pharmaceutically acceptable salts, Z¹ is C(R²).

In some embodiments of compounds of Formula (I), Formula (II), or their pharmaceutically acceptable salts, Y² is C(R²).

In some embodiments of compounds of Formula (I), Formula (II), or their pharmaceutically acceptable salts, Z² is C(R²).

In some embodiments of compounds of Formula (I), Formula (II), or their pharmaceutically acceptable salts, Y¹ is NR³.

In some embodiments of compounds of Formula (I), Formula (II), or their pharmaceutically acceptable salts, Z¹ is NR³.

In some embodiments of compounds of Formula (I), Formula (II), or their pharmaceutically acceptable salts, Y² is NR³.

In some embodiments of compounds of Formula (I), Formula (II), or their pharmaceutically acceptable salts, Z² is NR³.

In some embodiments of compounds of Formula (I), Formula (II), or their pharmaceutically acceptable salts, Y¹ is C(R²) and Z¹ is NR³. In some embodiments of compounds of Formula (I), Formula (II), or their pharmaceutically acceptable salts, Y² and Z² are C(R²). In some embodiments of compounds of Formula (I), Formula (II), or their pharmaceutically acceptable salts, Y² is C(R²) and Z² is NR³. In some embodiments of compounds of Formula (I), Formula (II), or their pharmaceutically acceptable salts, Y² is NR³ and Z² is C(R²). In some embodiments of compounds of Formula (I), Formula (II), or their pharmaceutically acceptable salts, Y² and Z² are NR³.

In some embodiments of compounds of Formula (I), Formula (II), or their pharmaceutically acceptable salts, at least one R² is hydrogen. In some embodiments of compounds of Formula (I), Formula (II), or their pharmaceutically acceptable salts, at least one R² is C₁₋₈ alkyl. In some embodiments of compounds of Formula (I), Formula (II), or their pharmaceutically acceptable salts, at least one R² is absent.

In some embodiments of compounds of Formula (I), Formula (II), or their pharmaceutically acceptable salts, at least one R³ is hydrogen. In some embodiments of compounds of Formula (I), Formula (II), or their pharmaceutically acceptable salts, at least one R³ is C₁₋₈ alkyl. In some embodiments of compounds of Formula (I), Formula (II), or their pharmaceutically acceptable salts, at least one R³ is absent.

In some embodiments of compounds of Formula (I), Formula (II), or their pharmaceutically acceptable salts, X is (C(R⁴)₂)ₘ. In some embodiments of compounds of Formula (I), Formula (II), or their pharmaceutically acceptable salts, at least one R⁴ is hydrogen. In some embodiments of compounds of Formula (I), Formula (II), or their pharmaceutically acceptable salts, m is 1.

In some embodiments of compounds of Formula (I), Formula (II), or their pharmaceutically acceptable salts, X is (C═NR⁵). In some embodiments of compounds of Formula (I), Formula (II), or their pharmaceutically acceptable salts, R⁵ is hydrogen.

In some embodiments of compounds of Formula (I), Formula (II), or their pharmaceutically acceptable salts, X is (C═O). In some embodiments of compounds of Formula (I), Formula (II), or their pharmaceutically acceptable salts, X is (C═S). In some embodiments of compounds of Formula (I), Formula (II), or their pharmaceutically acceptable salts, X is O. In some embodiments of compounds of Formula (I), Formula (II), or their pharmaceutically acceptable salts, X is SO₂.

Some embodiments of compounds of Formula (I) include compounds having the structure of Formula (Ia):

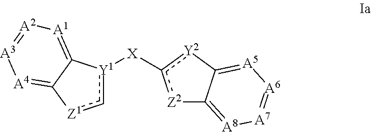

or a pharmaceutically acceptable salt thereof, wherein:
A¹, A², A³, A⁴, A⁵, A⁶, A⁷, and A⁸ are each independently C(R¹) or N, wherein at least one of A¹, A², A³, A⁴, A⁵, A⁶, A⁷, and A⁸ is C(R⁶) or N; and
Y¹, Y², Z¹, and Z² are each independently selected from the group consisting of O, S, C(R²), and NR³, wherein at least one of Y¹, Y², Z¹, and Z² is NR³.

In some embodiments of compounds of Formula (Ia) or their pharmaceutically acceptable salts; Y¹ is C(R²) and Z¹ is NR³. In some embodiments of compounds of Formula (Ia) or their pharmaceutically acceptable salts; Y² and Z² are C(R²). In some embodiments of compounds of Formula (Ia) or their pharmaceutically acceptable salts; Y² is C(R²) and Z² is NR³. In some embodiments of compounds of Formula (Ia) or their pharmaceutically acceptable salts; Y² is NR³ and Z² is C(R²). In some embodiments of compounds of Formula (Ia) or their pharmaceutically acceptable salts; Y² and Z² are NR³.

In some embodiments of compounds of Formula (Ia) or their pharmaceutically acceptable salts; R² is hydrogen. In some embodiments of compounds of Formula (Ia) or their pharmaceutically acceptable salts; R² is absent.

In some embodiments of compounds of Formula (Ia) or their pharmaceutically acceptable salts; R³ is hydrogen. In some embodiments of compounds of Formula (Ia) or their pharmaceutically acceptable salts; R³ is absent.

In some embodiments of compounds of Formula (Ia) or their pharmaceutically acceptable salts; X is (C(R⁴)₂)ₘ. In some embodiments of compounds of Formula (Ia) or their pharmaceutically acceptable salts; at least one R⁴ is hydrogen. In some embodiments of compounds of Formula (Ia) or their pharmaceutically acceptable salts; m is 1.

In some embodiments of compounds of Formula (Ia) or their pharmaceutically acceptable salts; X is (C═O).

Some embodiments of compounds of Formula (I) include compounds having the structure of Formula (Ia-2):

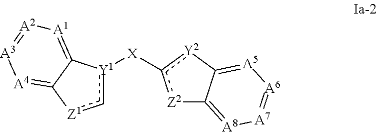

or a pharmaceutically acceptable salt thereof.

In some embodiments of compounds of Formula (Ia-2) or their pharmaceutically acceptable salts; Z¹ is NR³. In some embodiments of compounds of Formula (Ia-2) or their pharmaceutically acceptable salts; Y² is C(R²) and Z² is NR³. In some embodiments of compounds of Formula (Ia) or their pharmaceutically acceptable salts; Y² is NR³ and Z² is C(R²). In some embodiments of compounds of Formula (Ia) or their pharmaceutically acceptable salts; Y² and Z² are NR³.

In some embodiments of compounds of Formula (Ia) or their pharmaceutically acceptable salts; at least one R³ is hydrogen. In some embodiments of compounds of Formula (Ia) or their pharmaceutically acceptable salts; at least one R³ is absent.

Some embodiments of compounds of Formula (I) include compounds having the structure of Formula (Ia-3):

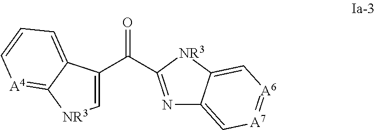

or a pharmaceutically acceptable salt thereof.

In some embodiments of compounds of Formula (Ia-3) or their pharmaceutically acceptable salts; A⁴ is C(R¹).

In some embodiments of compounds of Formula (Ia-3) or their pharmaceutically acceptable salts; A⁶ is C(R¹).

In some embodiments of compounds of Formula (Ia-3) or their pharmaceutically acceptable salts; A⁷ is C(R¹).

In some embodiments of compounds of Formula (Ia-3) or their pharmaceutically acceptable salts; at least one R¹ is hydrogen. In some embodiments of compounds of Formula (Ia-3) or their pharmaceutically acceptable salts; at least one R¹ is halo. In some embodiments of compounds of Formula (Ia-3) or their pharmaceutically acceptable salts; at least one R¹ is F. In some embodiments of compounds of Formula (Ia-3) or their pharmaceutically acceptable salts; at least one R¹ is Cl. In some embodiments of compounds of Formula (Ia-3) or their pharmaceutically acceptable salts; at least one R¹ is Br. In some embodiments of compounds of Formula (Ia-3) or their pharmaceutically acceptable salts; at least one R¹ is C₁₋₈ alkyl. In some embodiments of compounds of Formula (Ia-3) or their pharmaceutically acceptable salts; at least one R¹ is methyl. In some embodiments of compounds of Formula (Ia-3) or their pharmaceutically acceptable salts; at least one R¹ is C₁₋₈ haloalkyl. In some embodiments of compounds of Formula (Ia-3) or their pharmaceutically acceptable salts; at least one R¹ is CF₃.

In some embodiments of compounds of Formula (Ia-3) or their pharmaceutically acceptable salts; at least one R³ is hydrogen. In some embodiments of compounds of Formula (Ia-3) or their pharmaceutically acceptable salts; at least one R³ is C₁₋₈ alkyl. In some embodiments of compounds of Formula (Ia-3) or their pharmaceutically acceptable salts; at least one R³ is absent.

In some embodiments of compounds of Formula (Ia-3) or their pharmaceutically acceptable salts; at least one R⁶ is C₁₋₈ haloalkyl. In some embodiments of compounds of Formula (Ia-3) or their pharmaceutically acceptable salts; at least one R⁶ is CF₃.

Some embodiments of compounds of Formula (I) include compounds having the structure selected from:

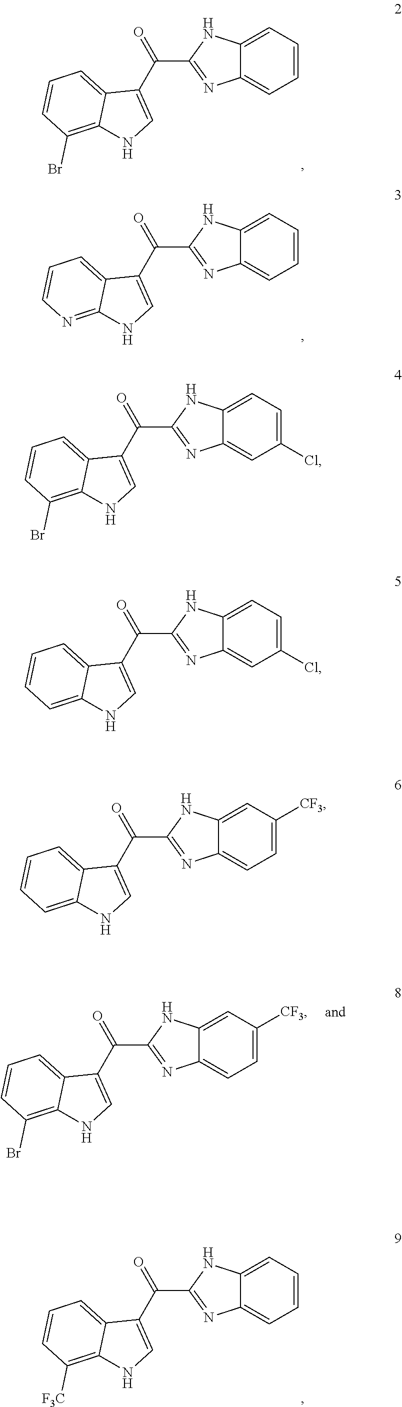

or a pharmaceutically acceptable salt thereof.

Some embodiments of compounds of Formula (II) having a structure selected from:

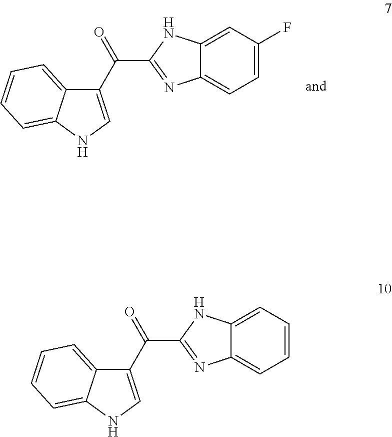 [SMILES: O=C(c1nc2ccc(F)cc2[nH]1)c1c[nH]c2ccccc12 ; O=C(c1nc2ccccc2[nH]1)c1c[nH]c2ccccc12]

or a pharmaceutically acceptable salt thereof.

Where the compounds disclosed herein have at least one chiral center, they may exist as individual enantiomers and diastereomers or as mixtures of such isomers, including racemates. Separation of the individual isomers or selective synthesis of the individual isomers is accomplished by application of various methods which are well known to practitioners in the art. Unless otherwise indicated, all such isomers and mixtures thereof are included in the scope of the compounds disclosed herein. Furthermore, compounds disclosed herein may exist in one or more crystalline or amorphous forms. Unless otherwise indicated, all such forms are included in the scope of the compounds disclosed herein including any polymorphic forms. In addition, some of the compounds disclosed herein may form solvates with water (i.e., hydrates) or common organic solvents. Unless otherwise indicated, such solvates are included in the scope of the compounds disclosed herein.

The skilled artisan will recognize that some structures described herein may be resonance forms or tautomers of compounds that may be fairly represented by other chemical structures, even when kinetically; the artisan recognizes that such structures may only represent a very small portion of a sample of such compound(s). Such compounds are considered within the scope of the structures depicted, though such resonance forms or tautomers are not represented herein.

### Isotopically-Labeled Compounds

Isotopes may be present in the compounds described. Each chemical element as represented in a compound structure may include any isotope of said element. The isotopes may be isotopes of carbon, chlorine, fluorine, hydrogen, iodine, nitrogen, oxygen, Phosphorous, sulfur, and technetium, including ¹¹C, ¹³C, ¹⁴C, ³⁶Cl, ¹⁸F, ²H, ³H, ¹²³I, ¹²⁵I, ¹³N, ¹⁵N, ¹⁵O, ¹⁷O, ¹⁸O, ³¹P, ³²P, ³⁵S, and ^{99m}Tc. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

Isotopically-labeled compounds of the present embodiments are useful in drug and substrate tissue distribution and target occupancy assays. For example, isotopically labeled compounds are particularly useful in SPECT (single photon emission computed tomography) and in PET (positron emission tomography).

### Definitions

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

“Solvate” refers to the compound formed by the interaction of a solvent and a compound described herein or salt thereof. Suitable solvates are pharmaceutically acceptable solvates including hydrates.

The term “pharmaceutically acceptable salt” refers to salts that retain the biological effectiveness and properties of a compound, which are not biologically or otherwise undesirable for use in a pharmaceutical. In many cases, the compounds herein are capable of forming acid and/or base salts by virtue of the presence of amino and/or carboxyl groups or groups similar thereto. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like; particularly preferred are the ammonium, potassium, sodium, calcium and magnesium salts. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. Many such salts are known in the art, as described in WO 87/05297, Johnston et al., published Sep. 11, 1987 (incorporated by reference herein in its entirety).

As used herein, “Cₐ to C_{b}” or “C_{a-b}” in which “a” and “b” are integers refer to the number of carbon atoms in the specified group. That is, the group can contain from “a” to “b”, inclusive, carbon atoms. Thus, for example, a “C₁ to C₄ alkyl” or “C₁₋₄ alkyl” group refers to all alkyl groups having from 1 to 4 carbons, that is, CH₃—, CH₃CH₂—, CH₃CH₂CH₂—, (CH₃)₂CH—, CH₃CH₂CH₂CH₂—, CH₃CH₂CH(CH₃)— and (CH₃)₃C—.

The term “halogen” or “halo,” as used herein, means any one of the radio-stable atoms of column 7 of the Periodic Table of the Elements, e.g., fluorine, chlorine, bromine, or iodine, with fluorine and chlorine being preferred.

As used herein, “alkyl” refers to a straight or branched hydrocarbon chain that is fully saturated (i.e., contains no double or triple bonds). The alkyl group may have 1 to 20 carbon atoms (whenever it appears herein, a numerical range such as “1 to 20” refers to each integer in the given range; e.g., “1 to 20 carbon atoms” means that the alkyl group may consist of 1 carbon atom, 2 carbon atoms, 3 carbon atoms, etc., up to and including 20 carbon atoms, although the present definition also covers the occurrence of the term “alkyl” where no numerical range is designated). The alkyl group may also be a medium size alkyl having 1 to 9 carbon atoms. The alkyl group could also be a lower alkyl having 1 to 4 carbon atoms. The alkyl group of the compounds may be designated as “C₁₋₄ alkyl” or similar designations. By way of example only, “C₁₋₄ alkyl” indicates that there are one to four carbon atoms in the alkyl chain, i.e., the alkyl chain is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, tertiary butyl, pentyl, hexyl, and the like.

As used herein, “haloalkyl” refers to a straight- or branched-chain alkyl group having from 1 to 12 carbon atoms in the chain, substituting one or more hydrogens with halogens. Examples of haloalkyl groups include, but are not limited to, —CF₃, —CHF₂, —CH₂F, —CH₂CF₃, —CH₂CHF₂, —CH₂CH₂F, —CH₂CH₂Cl, —CH₂CF₂CF₃ and other groups that in light of the ordinary skill in the art and the teachings provided herein, would be considered equivalent to any one of the foregoing examples.

As used herein, “alkoxy” refers to the formula-OR wherein R is an alkyl as is defined above, such as “C₁₋₉ alkoxy”, including but not limited to methoxy, ethoxy, n-propoxy, 1-methylethoxy (isopropoxy), n-butoxy, iso-butoxy, sec-butoxy, and tert-butoxy, and the like.

As used herein, “polyethylene glycol” refers to the formula

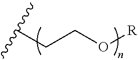

wherein n is an integer greater than one and R is a hydrogen or alkyl. The number of repeat units “n” may be indicated by referring to a number of members. Thus, for example, “2- to 5-membered polyethylene glycol” refers to n being an integer selected from two to five. In some embodiments, R is selected from methoxy, ethoxy, n-propoxy, 1-methylethoxy (isopropoxy), n-butoxy, iso-butoxy, sec-butoxy, and tert-butoxy.

As used herein, “heteroalkyl” refers to a straight or branched hydrocarbon chain containing one or more heteroatoms, that is, an element other than carbon, including but not limited to, nitrogen, oxygen and sulfur, in the chain backbone. The heteroalkyl group may have 1 to 20 carbon atoms although the present definition also covers the occurrence of the term “heteroalkyl” where no numerical range is designated. The heteroalkyl group may also be a medium size heteroalkyl having 1 to 9 carbon atoms. The heteroalkyl group could also be a lower heteroalkyl having 1 to 4 carbon atoms. In various embodiments, the heteroalkyl may have from 1 to 4 heteroatoms, from 1 to 3 heteroatoms, 1 or 2 heteroatoms, or 1 heteroatom. The heteroalkyl group of the compounds may be designated as “C₁₋₄ heteroalkyl” or similar designations. The heteroalkyl group may contain one or more heteroatoms. By way of example only, “C₁₋₄ heteroalkyl” indicates that there are one to four carbon atoms in the heteroalkyl chain and additionally one or more heteroatoms in the backbone of the chain.

The term “aromatic” refers to a ring or ring system having a conjugated pi electron system and includes both carbocyclic aromatic (e.g., phenyl) and heterocyclic aromatic groups (e.g., pyridine). The term includes monocyclic or fused-ring polycyclic (i.e., rings which share adjacent pairs of atoms) groups provided that the entire ring system is aromatic.

As used herein, “aryl” refers to an aromatic ring or ring system (i.e., two or more fused rings that share two adjacent carbon atoms) containing only carbon in the ring backbone. When the aryl is a ring system, every ring in the system is aromatic. The aryl group may have 6 to 18 carbon atoms, although the present definition also covers the occurrence of the term “aryl” where no numerical range is designated. In some embodiments, the aryl group has 6 to 10 carbon atoms. The aryl group may be designated as “C₆₋₁₀ aryl,” “C₆ or C₁₀ aryl,” or similar designations. Examples of aryl groups include, but are not limited to, phenyl, naphthyl, azulenyl, and anthracenyl.

As used herein, “aryloxy” and “arylthio” refers to RO— and RS—, in which R is an aryl as is defined above, such as “C₆₋₁₀ aryloxy” or “C₆₋₁₀ arylthio” and the like, including but not limited to phenyloxy.

An “aralkyl” or “arylalkyl” is an aryl group connected, as a substituent, via an alkylene group, such “C₇₋₁₄ aralkyl” and the like, including but not limited to benzyl, 2-phenylethyl, 3-phenylpropyl, and naphthylalkyl. In some cases, the alkylene group is a lower alkylene group (i.e., a C₁₋₄ alkylene group).

As used herein, “heteroaryl” refers to an aromatic ring or ring system (i.e., two or more fused rings that share two adjacent atoms) that contain(s) one or more heteroatoms, that is, an element other than carbon, including but not limited to, nitrogen, oxygen and sulfur, in the ring backbone. When the heteroaryl is a ring system, every ring in the system is aromatic. The heteroaryl group may have 5-18 ring members (i.e., the number of atoms making up the ring backbone, including carbon atoms and heteroatoms), although the present definition also covers the occurrence of the term “heteroaryl” where no numerical range is designated. In some embodiments, the heteroaryl group has 5 to 10 ring members or 5 to 7 ring members. The heteroaryl group may be designated as “5-7 membered heteroaryl,” “5-10 membered heteroaryl,” or similar designations. In various embodiments, a heteroaryl contains from 1 to 4 heteroatoms, from 1 to 3 heteroatoms, from 1 to 2 heteroatoms, or 1 heteroatom. For example, in various embodiments, a heteroaryl contains 1 to 4 nitrogen atoms, 1 to 3 nitrogen atoms, 1 to 2 nitrogen atoms, 2 nitrogen atoms and 1 sulfur or oxygen atom, 1 nitrogen atom and 1 sulfur or oxygen atom, or 1 sulfur or oxygen atom. Examples of heteroaryl rings include, but are not limited to, furyl, thienyl, phthalazinyl, pyrrolyl, oxazolyl, thiazolyl, imidazolyl, pyrazolyl, isoxazolyl, isothiazolyl, triazolyl, thiadiazolyl, pyridinyl, pyridazinyl, pyrimidinyl, pyrazinyl, triazinyl, quinolinyl, isoquinlinyl, benzimidazolyl, benzoxazolyl, benzothiazolyl, indolyl, isoindolyl, and benzothienyl.

A “heteroaralkyl” or “heteroarylalkyl” is heteroaryl group connected, as a substituent, via an alkylene group. Examples include but are not limited to 2-thienylmethyl, 3-thienylmethyl, furylmethyl, thienylethyl, pyrrolylalkyl, pyridylalkyl, isoxazollylalkyl, and imidazolylalkyl. In some cases, the alkylene group is a lower alkylene group (i.e., a C₁₋₄ alkylene group).

As used herein, “carbocyclyl” means a non-aromatic cyclic ring or ring system containing only carbon atoms in the ring system backbone. When the carbocyclyl is a ring system, two or more rings may be joined together in a fused, bridged or spiro-connected fashion. Carbocyclyls may have any degree of saturation provided that at least one ring in a ring system is not aromatic. Thus, carbocyclyls include cycloalkyls, cycloalkenyls, and cycloalkynyls. The carbocyclyl group may have 3 to 20 carbon atoms, although the present definition also covers the occurrence of the term “carbocyclyl” where no numerical range is designated. The carbocyclyl group may also be a medium size carbocyclyl having 3 to 10 carbon atoms. The carbocyclyl group could also be a carbocyclyl having 3 to 6 carbon atoms. The carbocyclyl group may be designated as “C₃₋₆ carbocyclyl” or similar Examples of carbocyclyl rings include, but are not limited to, cyclopropyl, designations, cyclobutyl, cyclopentyl, cyclohexyl, cyclohexenyl, 2,3-dihydro-indene, bicycle[2.2.2]octanyl, adamantyl, and spiro[4.4]nonanyl.

A “(carbocyclyl)alkyl” is a carbocyclyl group connected, as a substituent, via an alkylene group, such as “C₄₋₁₀ (carbocyclyl)alkyl” and the like, including but not limited to, cyclopropylmethyl, cyclobutylmethyl, cyclopropylethyl, cyclopropylbutyl, cyclobutylethyl, cyclopropylisopropyl, cyclopentylmethyl, cyclopentylethyl, cyclohexylmethyl, cyclohexylethyl, cycloheptylmethyl, and the like. In some cases, the alkylene group is a lower alkylene group.

As used herein, “cycloalkyl” means a fully saturated carbocyclyl ring or ring system. Examples include cyclopropyl, cyclobutyl, cyclopentyl, and cyclohexyl.

As used herein, “cycloalkenyl” means a carbocyclyl ring or ring system having at least one double bond, wherein no ring in the ring system is aromatic. An example is cyclohexenyl.

As used herein, “heterocyclyl” means a non-aromatic cyclic ring or ring system containing at least one heteroatom in the ring backbone. Heterocyclyls may be joined together in a fused, bridged or spiro-connected fashion. Heterocyclyls may have any degree of saturation provided that at least one ring in the ring system is not aromatic. The heteroatom(s) may be present in either a non-aromatic or aromatic ring in the ring system. The heterocyclyl group may have 3 to 20 ring members (i.e., the number of atoms making up the ring backbone, including carbon atoms and heteroatoms), although the present definition also covers the occurrence of the term “heterocyclyl” where no numerical range is designated. The heterocyclyl group may also be a medium size heterocyclyl having 3 to 10 ring members. The heterocyclyl group could also be a heterocyclyl having 3 to 6 ring members. The heterocyclyl group may be designated as “3-6 membered heterocyclyl” or similar designations.

In various embodiments, a heterocyclyl contains from 1 to 4 heteroatoms, from 1 to 3 heteroatoms, from 1 to 2 heteroatoms, or 1 heteroatom. For example, in various embodiments, a heterocyclyl contains 1 to 4 nitrogen atoms, 1 to 3 nitrogen atoms, 1 to 2 nitrogen atoms, 2 nitrogen atoms and 1 sulfur or oxygen atom, 1 nitrogen atom and 1 sulfur or oxygen atom, or 1 sulfur or oxygen atom. In preferred six membered monocyclic heterocyclyls, the heteroatom(s) are selected from one up to three of O, N or S, and in preferred five membered monocyclic heterocyclyls, the heteroatom(s) are selected from one or two heteroatoms selected from O, N, or S. Examples of heterocyclyl rings include, but are not limited to, azepinyl, acridinyl, carbazolyl, cinnolinyl, dioxolanyl, imidazolinyl, imidazolidinyl, morpholinyl, oxiranyl, oxepanyl, thiepanyl, piperidinyl, piperazinyl, dioxopiperazinyl, pyrrolidinyl, pyrrolidonyl, pyrrolidionyl, 4-piperidonyl, pyrazolinyl, pyrazolidinyl, 1,3-dioxinyl, 1,3-dioxanyl, 1,4-dioxinyl, 1,4-dioxanyl, 1,3-oxathianyl, 1,4-oxathiinyl, 1,4-oxathianyl, 2H-1,2-oxazinyl, trioxanyl, hexahydro-1,3,5-triazinyl, 1,3-dioxolyl, 1,3-dioxolanyl, 1,3-dithiolyl, 1,3-dithiolanyl, isoxazolinyl, isoxazolidinyl, oxazolinyl, oxazolidinyl, oxazolidinonyl, thiazolinyl, thiazolidinyl, 1,3-oxathiolanyl, indolinyl, isoindolinyl, tetrahydrofuranyl, tetrahydropyranyl, tetrahydrothiophenyl, tetrahydrothiopyranyl, tetrahydro-1,4-thiazinyl, thiamorpholinyl, dihydrobenzofuranyl, benzimidazolidinyl, and tetrahydroquinoline.

A “(heterocyclyl)alkyl” is a heterocyclyl group connected, as a substituent, via an alkylene group. Examples include, but are not limited to, imidazolinylmethyl and indolinylethyl.

As used herein, “acyl” refers to −C(═O)R, wherein R is hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein. Non-limiting examples include formyl, acetyl, propanoyl, benzoyl, and acryl.

An “O-carboxy” group refers to a “—OC(═O)R” group in which R is selected from hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein.

A “C-carboxy” group refers to a “—C(═O) OR” group in which R is selected from hydrogen, C₁₋₆ alkyl, C₂₋₆ alkenyl, C₂₋₆ alkynyl, C₃₋₇ carbocyclyl, aryl, 5-10 membered heteroaryl, and 5-10 membered heterocyclyl, as defined herein. A non-limiting example includes carboxyl (i.e., —C(═O) OH).

A “cyano” group refers to a “—CN” group.

A “cyanato” group refers to an “—OCN” group.

An “isocyanato” group refers to a “—NCO” group.

A “thiocyanato” group refers to a “—SCN” group.

An “isothiocyanato” group refers to an “—NCS” group.

A “sulfinyl” group refers to an “—S(—O)R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

A “sulfonyl” group refers to an “—SO₂R” group in which R is selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “S-sulfonamido” group refers to a “—SO₂NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-sulfonamido” group refers to a “—N(R_{A}) SO₂R_{B}” group in which R_{A} and R_{b} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “O-carbamyl” group refers to a “—OC(═O)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-carbamyl” group refers to an “—N(R_{A})OC(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “O-thiocarbamyl” group refers to a “—OC(═S)NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-thiocarbamyl” group refers to an “—N(R_{A})OC(═S)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

A “C-amido” group refers to a “—C(═O) NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “N-amido” group refers to a “—N(R_{A}) C(═O)R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “amino” group refers to a “—NR_{A}R_{B}” group in which R_{A} and R_{B} are each independently selected from hydrogen, C₁₋₆ alkyl, C₂₋₆ alkenyl, C₂₋₆ alkynyl, C₃₋₇ carbocyclyl, C₆₋₁₀ aryl, 5-10 membered heteroaryl, and 5-10 membered heterocyclyl, as defined herein.

An “aminoalkyl” group refers to an amino group connected via an alkylene group.

An “alkoxyalkyl” group refers to an alkoxy group connected via an alkylene group, such as a “C₂₋₈ alkoxyalkyl” and the like.

As used herein, a substituted group is derived from the unsubstituted parent group in which there has been an exchange of one or more hydrogen atoms for another atom or group. Unless otherwise indicated, when a group is deemed to be “substituted,” it is meant that the group is substituted with one or more substitutents independently selected from C₁-C₆ alkyl, C₁-C₆ alkenyl, C₁-C₆ alkynyl, C₁-C₆ heteroalkyl, C₃-C₇ carbocyclyl (optionally substituted with halo, C₁-C₆ alkyl, C₁-C₆ alkoxy, C₁-C₆ haloalkyl, and C₁-C₆ haloalkoxy), C₁-C₆-carbocyclyl-C₁-C₆-alkyl (optionally substituted with halo, C₁-C₆ alkyl, C₁-C₆ alkoxy, C₁-C₆ haloalkyl, and C₁-C₆ haloalkoxy), 5-10 membered heterocyclyl (optionally substituted with halo, C₁-C₆ alkyl, C₁-C₆ alkoxy, C₁-C₆ haloalkyl, and C₁-C₆ haloalkoxy), 5-10 membered heterocyclyl-C₁-C₆-alkyl (optionally substituted with halo, C₁-C₆ alkyl, C₁-C₆ alkoxy, C₁-C₆ haloalkyl, and C₁-C₆ haloalkoxy), aryl (optionally substituted with halo, C₁-C₆ alkyl, C₁-C₆ alkoxy, C₁-C₆ haloalkyl, and C₁-C₆ haloalkoxy), aryl(C₁-C₆)alkyl (optionally substituted with halo, C₁-C₆ alkyl, C₁-C₆ alkoxy, C₁-C₆ haloalkyl, and C₁-C₆ haloalkoxy), 5-10 membered heteroaryl (optionally substituted with halo, C₁-C₆ alkyl, C₁-C₆ alkoxy, C₁-C₆ haloalkyl, and C₁-C₆ haloalkoxy), 5-10 membered heteroaryl(C₁-C₆)alkyl (optionally substituted with halo, C₁-C₆ alkyl, C₁-C₆ alkoxy, C₁-C₆ haloalkyl, and C₁-C₆ haloalkoxy), halo, cyano, hydroxy, C₁-C₆ alkoxy, C₁-C₆ alkoxy (C₁-C₆)alkyl (i.e., ether), aryloxy, sulfhydryl(mercapto), halo(C₁-C₆)alkyl (e.g., —CF₃), halo(C₁-C₆)alkoxy (e.g., —OCF₃), C₁-C₆ alkylthio, arylthio, amino, amino (C₁-C₆)alkyl, nitro, O-carbamyl, N-carbamyl, O-thiocarbamyl, N-thiocarbamyl, C-amido, N-amido, S-sulfonamido, N-sulfonamido, C-carboxy, O-carboxy, acyl, cyanato, isocyanato, thiocyanato, isothiocyanato, sulfinyl, sulfonyl, and oxo (═O). Wherever a group is described as “optionally substituted” that group can be substituted with the above substituents.

In some embodiments, substituted group(s) is (are) substituted with one or more substituent(s) individually and independently selected from C₁-C₄ alkyl, amino, hydroxy, and halogen.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂, and the like. Other radical naming conventions clearly indicate that the radical is a di-radical such as “alkylene” or “alkenylene.”

When two R groups are said to form a ring (e.g., a carbocyclyl, heterocyclyl, aryl, or heteroaryl ring) “together with the atom to which they are attached,” it is meant that the collective unit of the atom and the two R groups are the recited ring. The ring is not otherwise limited by the definition of each R group when taken individually. For example, when the following substructure is present:

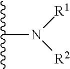

and R¹ and R² are defined as selected from the group consisting of hydrogen and alkyl, or R¹ and R² together with the nitrogen to which they are attached form a heterocyclyl, it is meant that R¹ and R² can be selected from hydrogen or alkyl, or alternatively, the substructure has structure:

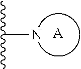

where ring A is a heterocyclyl ring containing the depicted nitrogen.

Similarly, when two “adjacent” R groups are said to form a ring “together with the atoms to which they are attached,” it is meant that the collective unit of the atoms, intervening bonds, and the two R groups are the recited ring. For example, when the following substructure is present:

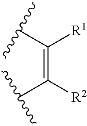

and R¹ and R² are defined as selected from the group consisting of hydrogen and alkyl, or R¹ and R² together with the atoms to which they are attached form an aryl or carbocyclyl, it is meant that R¹ and R² can be selected from hydrogen or alkyl, or alternatively, the substructure has structure:

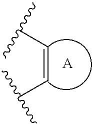

where A is an aryl ring or a carbocyclyl containing the depicted double bond.

Wherever a substituent is depicted as a di-radical (i.e., has two points of attachment to the rest of the molecule), it is to be understood that the substituent can be attached in any directional configuration unless otherwise indicated. Thus, for example, a substituent depicted as -AE- or

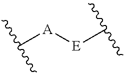

includes the substituent being oriented such that the A is attached at the leftmost attachment point of the molecule as well as the case in which A is attached at the rightmost attachment point of the molecule.

The term “agent” or “test agent” includes any substance, molecule, element, compound, entity, or a combination thereof. It includes, but is not limited to, e.g., protein, polypeptide, peptide or mimetic, small organic molecule, polysaccharide, polynucleotide, and the like. It can be a natural product, a synthetic compound, or a chemical compound, or a combination of two or more substances. Unless otherwise specified, the terms “agent”, “substance”, and “compound” are used interchangeably herein.

The term “mammal” is used in its usual biological sense. Thus, it specifically includes, but is not limited to, primates, including simians (chimpanzees, apes, monkeys) and humans, cattle, horses, sheep, goats, swine, rabbits, dogs, cats, rats and mice but also includes many other species.

The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

“Subject” as used herein, means a human or a non-human mammal, e.g., a dog, a cat, a mouse, a rat, a cow, a sheep, a pig, a goat, a non-human primate or a bird, e.g., a chicken, as well as any other vertebrate or invertebrate.

An “effective amount” or a “therapeutically effective amount” as used herein refers to an amount of a therapeutic agent that is effective to relieve, to some extent, or to reduce the likelihood of onset of, one or more of the symptoms of a disease or condition, and includes curing a disease or condition. “Curing” means that the symptoms of a disease or condition are eliminated; however, certain long-term or permanent effects may exist even after a cure is obtained (such as extensive tissue damage).

“Treat,” “treatment,” or “treating,” as used herein refers to administering a pharmaceutical composition for prophylactic and/or therapeutic purposes. The term “prophylactic treatment” refers to treating a subject who does not yet exhibit symptoms of a disease or condition, but who is susceptible to, or otherwise at risk of, a particular disease or condition, whereby the treatment reduces the likelihood that the patient will develop the disease or condition. The term “therapeutic treatment” refers to administering treatment to a subject who exhibits symptoms of a disease or condition, whereby the treatment reduces the symptoms of the disease or condition.

“Aryl hydrocarbon receptor (AhR) signaling” as used herein refers to the activation of signaling pathways by the aryl hydrocarbon receptor. Signaling, includes, but is not limited to, induction of target genes transcription and production of proteins. Specific target proteins effected by AhR signaling include Th2 and Th17 as well as IL-4, IL-13, IL-31, thymic stromal lymphopoietin, IL-5, or IL-33, IL-17, IL-22, IL-23, IL-12, IL-24, IL-26, IL-36, CCL20, and cathelicidin. AhR signaling can include the effects caused by these target proteins, such as products subsequently produced in downstream pathways.

### Methods of Preparation

The compounds disclosed herein may be synthesized by methods described below, or by modification of these methods. Ways of modifying the methodology include, among others, temperature, solvent, reagents etc., known to those skilled in the art. In general, during any of the processes for preparation of the compounds disclosed herein, it may be necessary and/or desirable to protect sensitive or reactive groups on any of the molecules concerned. This may be achieved by means of conventional protecting groups, such as those described in Protective Groups in Organic Chemistry (ed. J. F. W. McOmie, Plenum Press, 1973); and P. G. M. Green, T. W. Wutts, Protecting Groups in Organic Synthesis (3rd ed.) Wiley, New York (1999), which are both hereby incorporated herein by reference in their entirety. The protecting groups may be removed at a convenient subsequent stage using methods known from the art. Synthetic chemistry transformations useful in synthesizing applicable compounds are known in the art and include e.g. those described in R. Larock, Comprehensive Organic Transformations, VCH Publishers, 1989, or L. Paquette, ed., Encyclopedia of Reagents for Organic Synthesis, John Wiley and Sons, 1995, which are both hereby incorporated herein by reference in their entirety. The routes shown and described herein are illustrative only and are not intended, nor are they to be construed, to limit the scope of the claims in any manner whatsoever. Those skilled in the art will be able to recognize modifications of the disclosed syntheses and to devise alternate routes based on the disclosures herein; all such modifications and alternate routes are within the scope of the claims.

In the following schemes, protecting groups for oxygen atoms are selected for their compatibility with the requisite synthetic steps as well as compatibility of the introduction and deprotection steps with the overall synthetic schemes (P. G. M. Green, T. W. Wutts, Protecting Groups in Organic Synthesis (3rd ed.) Wiley, New York (1999)).

If the compounds of the present technology contain one or more chiral centers, such compounds can be prepared or isolated as pure stereoisomers, i.e., as individual enantiomers or d(l) stereoisomers, or as stereoisomer-enriched mixtures. All such stereoisomers (and enriched mixtures) are included within the scope of the present technology, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

The starting materials for the following reactions are generally known compounds or can be prepared by known procedures or obvious modifications thereof. For example, many of the starting materials are available from commercial suppliers such as Aldrich Chemical Co. (Milwaukee, Wisconsin, USA), Bachem (Torrance, California, USA), Emka-Chemce or Sigma (St. Louis, Missouri, USA). Others may be prepared by procedures, or obvious modifications thereof, described in standard reference texts such as Fieser and Fieser's Reagents for Organic Synthesis, Volumes 1-15 (John Wiley, and Sons, 1991), Rodd's Chemistry of Carbon Compounds, Volumes 1-5, and Supplementals (Elsevier Science Publishers, 1989), Organic Reactions, Volumes 1-40 (John Wiley, and Sons, 1991), March's Advanced Organic Chemistry, (John Wiley, and Sons, 5th Edition, 2001), and Larock's Comprehensive Organic Transformations (VCH Publishers Inc., 1989).

### Synthesis of Compounds of Formula I or II

In one embodiment, the method involves reacting an appropriately substituted reagent (IIIa) with the chloro-containing reagent (IIIb) in the presence of aluminum chloride to yield the desired compound of Formula (I). (Scheme 1).

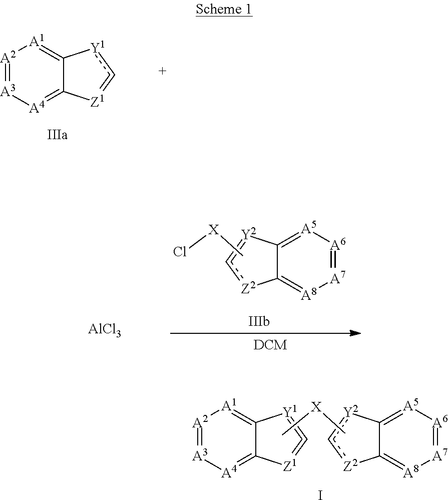

In another embodiment, the method involves reacting an appropriately substituted reagent (IVa) with the chloro-containing reagent (IVb) in the presence of aluminum chloride to yield the desired compound of Formula (II). (Scheme 2).

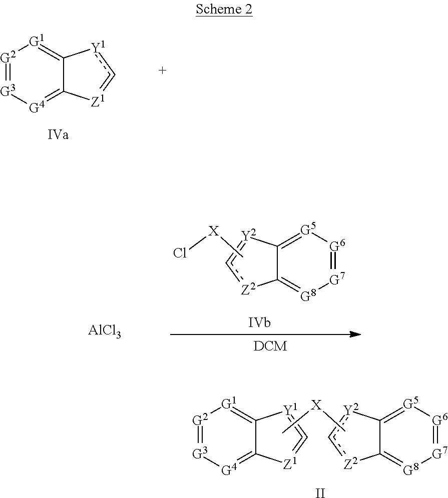

The above example scheme(s) are provided for the guidance of the reader, and collectively represent an example method for making the compounds encompassed herein. Furthermore, other methods for preparing compounds described herein will be readily apparent to the person of ordinary skill in the art in light of the following reaction schemes and examples. Unless otherwise indicated, all variables are as defined above.

### Administration and Pharmaceutical Compositions

The compounds are administered at a therapeutically effective dosage. While human dosage levels have yet to be optimized for the compounds described herein, generally, a daily dose may be from about 0.25 mg/kg to about 120 mg/kg or more of body weight, from about 0.5 mg/kg or less to about 70 mg/kg, from about 1.0 mg/kg to about 50 mg/kg of body weight, or from about 1.5 mg/kg to about 10 mg/kg of body weight. Thus, for administration to a 70 kg person, the dosage range would be from about 17 mg per day to about 8000 mg per day, from about 35 mg per day or less to about 7000 mg per day or more, from about 70 mg per day to about 6000 mg per day, from about 100 mg per day to about 5000 mg per day, or from about 200 mg to about 3000 mg per day. The amount of active compound administered will, of course, be dependent on the subject and disease state being treated, the severity of the affliction, the manner and schedule of administration and the judgment of the prescribing physician.

Administration of the compounds disclosed herein or the pharmaceutically acceptable salts thereof can be via any of the accepted modes of administration for agents that serve similar utilities including, but not limited to, orally, subcutaneously, intravenously, intranasally, topically, transdermally, intraperitoneally, intramuscularly, intrapulmonarilly, vaginally, rectally, or intraocularly. Oral and parenteral administrations are customary in treating the indications that are the subject of the preferred embodiments.

The compounds useful as described above can be formulated into pharmaceutical compositions for use in treatment of these conditions. Standard pharmaceutical formulation techniques are used, such as those disclosed in Remington's The Science and Practice of Pharmacy, 21st Ed., Lippincott Williams & Wilkins (2005), incorporated by reference in its entirety. Accordingly, some embodiments include pharmaceutical compositions comprising: (a) a safe and therapeutically effective amount of a compound described herein (including enantiomers, diastereoisomers, tautomers, polymorphs, and solvates thereof), or pharmaceutically acceptable salts thereof; and (b) a pharmaceutically acceptable carrier, diluent, excipient or combination thereof.

In addition to the selected compound useful as described above, some embodiments include compositions containing a pharmaceutically-acceptable carrier. The term “pharmaceutically acceptable carrier” or “pharmaceutically acceptable excipient” includes any and all solvents, dispersion media, coatings, antibacterial and antifungal agents, isotonic and absorption delaying agents and the like. The use of such media and agents for pharmaceutically active substances is well known in the art. Except insofar as any conventional media or agent is incompatible with the active ingredient, its use in the therapeutic compositions is contemplated. In addition, various adjuvants such as are commonly used in the art may be included. Considerations for the inclusion of various components in pharmaceutical compositions are described, e.g., in Gilman et al. (Eds.) (1990); Goodman and Gilman's: The Pharmacological Basis of Therapeutics, 8th Ed., Pergamon Press, which is incorporated herein by reference in its entirety.

Some examples of substances, which can serve as pharmaceutically-acceptable carriers or components thereof, are sugars, such as lactose, glucose and sucrose; starches, such as corn starch and potato starch; cellulose and its derivatives, such as sodium carboxymethyl cellulose, ethyl cellulose, and methyl cellulose; powdered tragacanth; malt; gelatin; talc; solid lubricants, such as stearic acid and magnesium stearate; calcium sulfate; vegetable oils, such as peanut oil, cottonseed oil, sesame oil, olive oil, corn oil and oil of theobroma; polyols such as propylene glycol, glycerine, sorbitol, mannitol, and polyethylene glycol; alginic acid; emulsifiers, such as the TWEENS; wetting agents, such sodium lauryl sulfate; coloring agents; flavoring agents; tableting agents, stabilizers; antioxidants; preservatives, pyrogen-free water; isotonic saline; and phosphate buffer solutions.

The choice of a pharmaceutically-acceptable carrier to be used in conjunction with the subject compound is basically determined by the way the compound is to be administered.

The compositions described herein are preferably provided in unit dosage form. As used herein, a “unit dosage form” is a composition containing an amount of a compound that is suitable for administration to an animal, preferably mammal subject, in a single dose, according to good medical practice. The preparation of a single or unit dosage form, however, does not imply that the dosage form is administered once per day or once per course of therapy. Such dosage forms are contemplated to be administered once, twice, thrice or more per day and may be administered as infusion over a period of time (e.g., from about 30 minutes to about 2-6 hours), or administered as a continuous infusion, and may be given more than once during a course of therapy, though a single administration is not specifically excluded. The skilled artisan will recognize that the formulation does not specifically contemplate the entire course of therapy and such decisions are left for those skilled in the art of treatment rather than formulation.

The compositions useful as described above may be in any of a variety of suitable forms for a variety of routes for administration, for example, for oral, nasal, rectal, topical (including transdermal), ocular, intracerebral, intracranial, intrathecal, intra-arterial, intravenous, intramuscular, or other parental routes of administration. The skilled artisan will appreciate that oral and nasal compositions comprise compositions that are administered by inhalation, and made using available methodologies. Depending upon the particular route of administration desired, a variety of pharmaceutically-acceptable carriers well-known in the art may be used. Pharmaceutically-acceptable carriers include, for example, solid or liquid fillers, diluents, hydrotropics, surface-active agents, and encapsulating substances. Optional pharmaceutically-active materials may be included, which do not substantially interfere with the inhibitory activity of the compound. The amount of carrier employed in conjunction with the compound is sufficient to provide a practical quantity of material for administration per unit dose of the compound. Techniques and compositions for making dosage forms useful in the methods described herein are described in the following references, all incorporated by reference herein: Modern Pharmaceutics, 4th Ed., Chapters 9 and 10 (Banker & Rhodes, editors, 2002); Lieberman et al., Pharmaceutical Dosage Forms: Tablets (1989); and Ansel, Introduction to Pharmaceutical Dosage Forms, 8th Edition (2004).

Various oral dosage forms can be used, including such solid forms as tablets, capsules, granules and bulk powders. Tablets can be compressed, tablet triturates, enteric-coated, sugar-coated, film-coated, or multiple-compressed, containing suitable binders, lubricants, diluents, disintegrating agents, coloring agents, flavoring agents, flow-inducing agents, and melting agents. Liquid oral dosage forms include aqueous solutions, emulsions, suspensions, solutions and/or suspensions reconstituted from non-effervescent granules, and effervescent preparations reconstituted from effervescent granules, containing suitable solvents, preservatives, emulsifying agents, suspending agents, diluents, sweeteners, melting agents, coloring agents and flavoring agents.

The pharmaceutically-acceptable carrier suitable for the preparation of unit dosage forms for peroral administration is well-known in the art. Tablets typically comprise conventional pharmaceutically-compatible adjuvants as inert diluents, such as calcium carbonate, sodium carbonate, mannitol, lactose and cellulose; binders such as starch, gelatin and sucrose; disintegrants such as starch, alginic acid and croscarmellose; lubricants such as magnesium stearate, stearic acid and talc. Glidants such as silicon dioxide can be used to improve flow characteristics of the powder mixture. Coloring agents, such as the FD&C dyes, can be added for appearance. Sweeteners and flavoring agents, such as aspartame, saccharin, menthol, peppermint, and fruit flavors, are useful adjuvants for chewable tablets. Capsules typically comprise one or more solid diluents disclosed above. The selection of carrier components depends on secondary considerations like taste, cost, and shelf stability, which are not critical, and can be readily made by a person skilled in the art.

Peroral compositions also include liquid solutions, emulsions, suspensions, and the like. The pharmaceutically-acceptable carriers suitable for preparation of such compositions are well known in the art. Typical components of carriers for syrups, elixirs, emulsions and suspensions include ethanol, glycerol, propylene glycol, polyethylene glycol, liquid sucrose, sorbitol and water. For a suspension, typical suspending agents include methyl cellulose, sodium carboxymethyl cellulose, AVICEL RC-591, tragacanth and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Such compositions may also be coated by conventional methods, typically with pH or time-dependent coatings, such that the subject compound is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, Eudragit coatings, waxes and shellac.

Compositions described herein may optionally include other drug actives.

Other compositions useful for attaining systemic delivery of the subject compounds include sublingual, buccal and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants and flavoring agents disclosed above may also be included.

A liquid composition, which is formulated for topical ophthalmic use, is formulated such that it can be administered topically to the eye. The comfort should be maximized as much as possible, although sometimes formulation considerations (e.g. drug stability) may necessitate less than optimal comfort. In the case that comfort cannot be maximized, the liquid should be formulated such that the liquid is tolerable to the patient for topical ophthalmic use. Additionally, an ophthalmically acceptable liquid should either be packaged for single use, or contain a preservative to prevent contamination over multiple uses.

For ophthalmic application, solutions or medicaments are often prepared using a physiological saline solution as a major vehicle. Ophthalmic solutions should preferably be maintained at a comfortable pH with an appropriate buffer system. The formulations may also contain conventional, pharmaceutically acceptable preservatives, stabilizers and surfactants.

Preservatives that may be used in the pharmaceutical compositions disclosed herein include, but are not limited to, benzalkonium chloride, PHMB, chlorobutanol, thimerosal, phenylmercuric, acetate and phenylmercuric nitrate. A useful surfactant is, for example, Tween 80. Likewise, various useful vehicles may be used in the ophthalmic preparations disclosed herein. These vehicles include, but are not limited to, polyvinyl alcohol, povidone, hydroxypropyl methyl cellulose, poloxamers, carboxymethyl cellulose, hydroxyethyl cellulose and purified water.

Tonicity adjustors may be added as needed or convenient. They include, but are not limited to, salts, particularly sodium chloride, potassium chloride, mannitol and glycerin, or any other suitable ophthalmically acceptable tonicity adjustor.

Various buffers and means for adjusting pH may be used so long as the resulting preparation is ophthalmically acceptable. For many compositions, the pH will be between 4 and 9. Accordingly, buffers include acetate buffers, citrate buffers, phosphate buffers and borate buffers. Acids or bases may be used to adjust the pH of these formulations as needed.

In a similar vein, an ophthalmically acceptable antioxidant includes, but is not limited to, sodium metabisulfite, sodium thiosulfate, acetylcysteine, butylated hydroxyanisole and butylated hydroxytoluene.

Other excipient components, which may be included in the ophthalmic preparations, are chelating agents. A useful chelating agent is edetate disodium, although other chelating agents may also be used in place or in conjunction with it.

For topical use, creams, ointments, gels, solutions or suspensions, etc., containing the compound disclosed herein are employed. Topical formulations may generally be comprised of a pharmaceutical carrier, co-solvent, emulsifier, penetration enhancer, preservative system, and emollient.

For intravenous administration, the compounds and compositions described herein may be dissolved or dispersed in a pharmaceutically acceptable diluent, such as a saline or dextrose solution. Suitable excipients may be included to achieve the desired pH, including but not limited to NaOH, sodium carbonate, sodium acetate, HCl, and citric acid. In various embodiments, the pH of the final composition ranges from 2 to 8, or preferably from 4 to 7. Antioxidant excipients may include sodium bisulfite, acetone sodium bisulfite, sodium formaldehyde, sulfoxylate, thiourea, and EDTA. Other non-limiting examples of suitable excipients found in the final intravenous composition may include sodium or potassium phosphates, citric acid, tartaric acid, gelatin, and carbohydrates such as dextrose, mannitol, and dextran. Further acceptable excipients are described in Powell, et al., Compendium of Excipients for Parenteral Formulations, PDA J Pharm Sci and Tech 1998, 52 238-311 and Nema et al., Excipients and Their Role in Approved Injectable Products: Current Usage and Future Directions, PDA J Pharm Sci and Tech 2011, 65 287-332, both of which are incorporated herein by reference in their entirety. Antimicrobial agents may also be included to achieve a bacteriostatic or fungistatic solution, including but not limited to phenylmercuric nitrate, thimerosal, benzethonium chloride, benzalkonium chloride, phenol, cresol, and chlorobutanol.

The compositions for intravenous administration may be provided to caregivers in the form of one more solids that are reconstituted with a suitable diluent such as sterile water, saline or dextrose in water shortly prior to administration. In other embodiments, the compositions are provided in solution ready to administer parenterally. In still other embodiments, the compositions are provided in a solution that is further diluted prior to administration. In embodiments that include administering a combination of a compound described herein and another agent, the combination may be provided to caregivers as a mixture, or the caregivers may mix the two agents prior to administration, or the two agents may be administered separately.

The actual dose of the active compounds described herein depends on the specific compound, and on the condition to be treated; the selection of the appropriate dose is well within the knowledge of the skilled artisan.

The compounds and compositions described herein, if desired, may be presented in a pack or dispenser device containing one or more unit dosage forms containing the active ingredient. Such a pack or device may, for example, comprise metal or plastic foil, such as a blister pack, or glass, and rubber stoppers such as in vials. The pack or dispenser device may be accompanied by instructions for administration. Compounds and compositions described herein are formulated in a compatible pharmaceutical carrier may also be prepared, placed in an appropriate container, and labeled for treatment of an indicated condition.

The amount of the compound in a formulation can vary within the full range employed by those skilled in the art. Typically, the formulation will contain, on a weight percent (wt %) basis, from about 0.01 to about 99.99 wt % of a compound of the present technology based on the total formulation, with the balance being one or more suitable pharmaceutical excipients. Preferably, the compound is present at a level of about 1 to about 80 wt %. Representative pharmaceutical formulations are described below.

### Formulation Examples

The following are representative pharmaceutical formulations containing a compound of Formula (I) or Formula (II).

### Formulation Example 1—Tablet Formulation

The following ingredients are mixed intimately and pressed into single scored tablets.

| | Quantity per |
|---|---|
| Ingredient | tablet, mg |
| Compounds disclosed herein | 400 |
| cornstarch | 50 |
| croscarmellose sodium | 25 |
| lactose | 120 |
| magnesium stearate | 5 |

### Formulation Example 2—Capsule Formulation

The following ingredients are mixed intimately and loaded into a hard-shell gelatin capsule.

| | Quantity per |
|---|---|
| Ingredient | capsule, mg |
| Compounds disclosed herein | 200 |
| lactose, spray-dried | 148 |
| magnesium stearate | 2 |

### Formulation Example 3—Suspension Formulation

The following ingredients are mixed to form a suspension for oral administration.

| Ingredient | Amount | |
|---|---|---|
| Compounds disclosed herein | 1.0 | g |
| fumaric acid | 0.5 | g |
| sodium chloride | 2.0 | g |
| methyl paraben | 0.15 | g |
| propyl paraben | 0.05 | g |
| granulated sugar | 25.0 | g |
| sorbitol (70% solution) | 13.00 | g |
| Veegum K (Vanderbilt Co.) | 1.0 | g |
| flavoring | 0.035 | mL |
| colorings | 0.5 | mg |

| | |
|---|---|
| distilled water | q.s. to 100 mL |

### Formulation Example 4—Injectable Formulation

The following ingredients are mixed to form an injectable formulation.

| Ingredient | Amount |
|---|---|
| Compounds disclosed herein | 0.2 mg-20 mg |
| sodium acetate buffer solution, 0.4M | 2.0 mL |
| HCl (1N) or NaOH (1N) | q.s. to suitable pH |
| water (distilled, sterile) | q.s. to 20 mL |

### Formulation Example 5—Suppository Formulation

A suppository of total weight 2.5 g is prepared by mixing the compound of the present technology with Witepsol® H-15 (triglycerides of saturated vegetable fatty acid; Riches-Nelson, Inc., New York), and has the following composition:

| Ingredient | Amount |
|---|---|
| Compounds disclosed herein | 500 mg |
| Witepsol ® H-15 | balance |

### Methods of Treatment

The compounds disclosed herein or their tautomers and/or pharmaceutically acceptable salts thereof can effectively act as aryl hydrocarbon receptor inhibitors and treat conditions mediated at least in part by aryl hydrocarbon receptor (AhR) signaling.

Some embodiments provide a method for treating a dermatologic disease with an effective amount of one or more compounds as disclosed herein, or a pharmaceutical composition comprising one or more of the compounds. Some embodiments provide a method for treating a respiratory disease with an effective amount of one or more compounds as disclosed herein, or a pharmaceutical composition comprising one or more of the compounds. Some embodiments provide a method for treating an oncologic disease with an effective amount of one or more compounds as disclosed herein, or a pharmaceutical composition comprising one or more of the compounds. Some embodiments provide a method for treating an autoimmune disease with an effective amount of one or more compounds as disclosed herein, or a pharmaceutical composition comprising one or more of the compounds. Some embodiments provide a method for treating an immunologic disease with an effective amount of one or more compounds as disclosed herein, or a pharmaceutical composition comprising one or more of the compounds.

Some embodiments provide a method for inhibiting aryl hydrocarbon receptor and/or a method for treating a disease mediated at least in part by AhR with an effective amount of one or more compounds as disclosed herein, or a pharmaceutical composition comprising one or more of the compounds.

Some embodiments provide a method for inhibiting AhR, which method comprises contacting cells with an effective amount of one or more compounds as disclosed herein. In some embodiments, the cells are epithelial, endothelial or stromal cells in the skin.

Some embodiments provide a method for treating a dermatologic disease, a respiratory disease, an oncologic disease, an autoimmune disease, or an immunologic disease, which method comprises administering to a subject an effective amount of one or more compounds or a pharmaceutical composition disclosed herein comprising a pharmaceutically acceptable excipient. In some embodiments, the compound is a compound of Formula (I). In other embodiments, the compound is a compound of Formula (II).

Some embodiments provide a method for prophylactic or therapeutic treatment of a subject having a dermatologic disease wherein said method comprising administering an effective amount of one or more compounds of Formula (I) or Formula (II) to the subject in need thereof.

Some embodiments provide a method for prophylactic or therapeutic treatment of a subject having a dermatologic disease mediated by AhR signaling wherein said method comprising administering an effective amount of one or more compounds of Formula (I) or Formula (II) to the subject in need thereof.

Some embodiments provide a method for treating a dermatologic disease selected from the group consisting of, or that produces a symptom selected from the group consisting of: atopic dermatitis, eczema, psoriasis, contact dermatitis, vitiligo, hidradenitis suppurativa, lichen planus, ichthyosis, palmoplantar pustulosis, dermatomyositis, netherton's syndrome, fibrosis, scleroderma, basal cell carcinoma, squamous cell carcinoma, merkel cell carcinoma, skin aging, bullous pemphigoid, sarcoidosis, chronic urticaria, rosacea, alopecia areata, pyoderma gangrenosum, cutaneous T cell lymphoma, or any combination thereof.

Some embodiments provide a method for prophylactic or therapeutic treatment of a subject having a respiratory disease wherein said method comprising administering an effective amount of one or more compounds of Formula (I) or Formula (II) to the subject in need thereof.

Some embodiments provide a method for prophylactic or therapeutic treatment of a subject having a respiratory disease mediated by AhR signaling wherein said method comprising administering an effective amount of one or more compounds of Formula (I) or Formula (II) to the subject in need thereof.

Some embodiments provide a method for treating a respiratory disease selected from the group consisting of, or that produces a symptom selected from the group consisting of: asthma, chronic obstructive pulmonary disorder, idiopathic pulmonary fibrosis, bronchitis, cystic fibrosis, acute respiratory distress syndrome, interstitial lung disease, or any combination thereof.

Some embodiments provide a method for prophylactic or therapeutic treatment of a subject having an oncologic disease wherein said method comprising administering an effective amount of one or more compounds of Formula (I) or Formula (II) to the subject in need thereof.

Some embodiments provide a method for prophylactic or therapeutic treatment of a subject having an oncologic disease mediated by AhR signaling wherein said method comprising administering an effective amount of one or more compounds of Formula (I) or Formula (II) to the subject in need thereof.

Some embodiments provide a method for treating an oncologic disease selected from the group consisting of, or that produces a symptom selected from the group consisting of: melanoma, non-small cell lung cancer, prostate cancer, pancreatic cancer, bladder cancer, mesothelioma, or any combination thereof.

Some embodiments provide a method for prophylactic or therapeutic treatment of a subject having an autoimmune disease wherein said method comprising administering an effective amount of one or more compounds of Formula (I) or Formula (II) to the subject in need thereof.

Some embodiments provide a method for prophylactic or therapeutic treatment of a subject having an autoimmune disease mediated by AhR signaling wherein said method comprising administering an effective amount of one or more compounds of Formula (I) or Formula (II) to the subject in need thereof.

Some embodiments provide a method for treating an autoimmune disease selected from the group consisting of, or that produces a symptom selected from the group consisting of: ankylosing spondylitis, rheumatoid arthritis, psoriatic arthritis, systemic lupus erythematosus, diabetic nephropathy, Graves' disease, multiple sclerosis, type 1 diabetes, Sjogren's disease, neuromyelitis optica, amyloidosis, IgG4 disease, Behcet's disease, Celiac disease, fibromyalgia, ulcerative colitis, Crohn's disease, uveitis, or any combination thereof.

Some embodiments provide a method for prophylactic or therapeutic treatment of a subject having an immunologic disease wherein said method comprising administering an effective amount of one or more compounds of Formula (I) or Formula (II) to the subject in need thereof.

Some embodiments provide a method for prophylactic or therapeutic treatment of a subject having an immunologic disease mediated AhR signaling wherein said method comprising administering an effective amount of one or more compounds of Formula (I) or Formula (II) to the subject in need thereof.

Some embodiments provide a method for treating an immunologic disease selected from the group consisting of, or that produces a symptom selected from the group consisting of: COVID-19, SARS-COV2, graft versus host disease, cytokine release syndrome, STAT3 gain of function, interferonopathies, Kawasaki disease, multi-inflammatory syndrome, or any combination thereof.

Some embodiments provide a method for treating a disease, the disease selected from: a dermatologic disease selected from hidradenitis suppurativa, lichen planus, ichthyosis, palmoplantar pustulosis, dermatomyositis, netherton's syndrome, fibrosis, scleroderma, bullous pemphigoid, chronic urticaria, rosacea, alopecia areata, pyoderma gangrenosum, or any combination thereof; an autoimmune disease selected from diabetic nephropathy, Graves' disease, neuromyelitis optica, IgG4 disease, Celiac disease, fibromyalgia, uveitis, or any combination thereof; a respiratory disease selected from idiopathic pulmonary fibrosis, cystic fibrosis, acute respiratory distress syndrome, interstitial lung disease, or any combination thereof, an immunologic disease selected from COVID-19, SARS-COV2, cytokine release syndrome, STAT3 gain of function, interferonopathies, multi-inflammatory syndrome, or any combination thereof; the method comprising administering to a subject in need thereof, a compound having the structure of the formula (II).

Some embodiments provide a method for treating a dermatologic disease, which method comprises administering to a subject an effective amount of one or more compounds of Formula (II) or a pharmaceutical composition disclosed herein comprising a pharmaceutically acceptable excipient. In some embodiments, the dermatologic disease is selected from the group consisting of, or that produces a symptom selected from the group consisting of: hidradenitis suppurativa, lichen planus, ichthyosis, palmoplantar pustulosis, dermatomyositis, netherton's syndrome, fibrosis, scleroderma, bullous pemphigoid, chronic urticaria, rosacea, alopecia areata, pyoderma gangrenosum, or any combination thereof.

Some embodiments provide a method for treating a respiratory disease, which method comprises administering to a subject an effective amount of one or more compounds of Formula (II) or a pharmaceutical composition disclosed herein comprising a pharmaceutically acceptable excipient. In some embodiments, the respiratory disease is selected from the group consisting of, or that produces a symptom selected from the group consisting of: idiopathic pulmonary fibrosis, cystic fibrosis, acute respiratory distress syndrome, interstitial lung disease, or any combination thereof.

Some embodiments provide a method for treating an autoimmune disease, which method comprises administering to a subject an effective amount of one or more compounds of Formula (II) or a pharmaceutical composition disclosed herein comprising a pharmaceutically acceptable excipient. In some embodiments, the autoimmune disease is selected from the group consisting of, or that produces a symptom selected from the group consisting of: diabetic nephropathy, Graves' disease, neuromyelitis optica, IgG4 disease, Celiac disease, fibromyalgia, uveitis, or any combination thereof.

Some embodiments provide a method for treating an immunologic disease, which method comprises administering to a subject an effective amount of one or more compounds of Formula (II) or a pharmaceutical composition disclosed herein comprising a pharmaceutically acceptable excipient. In some embodiments, the immunologic disease is selected from the group consisting of, or that produces a symptom selected from the group consisting of: COVID-19, SARS-COV2, cytokine release syndrome, STAT3 gain of function, interferonopathies, multi-inflammatory syndrome, or any combination thereof.

Some embodiments provide a method for administering to a subject an effective amount of one or more compounds of Formula (I) or Formula (II) having the structure selected from:

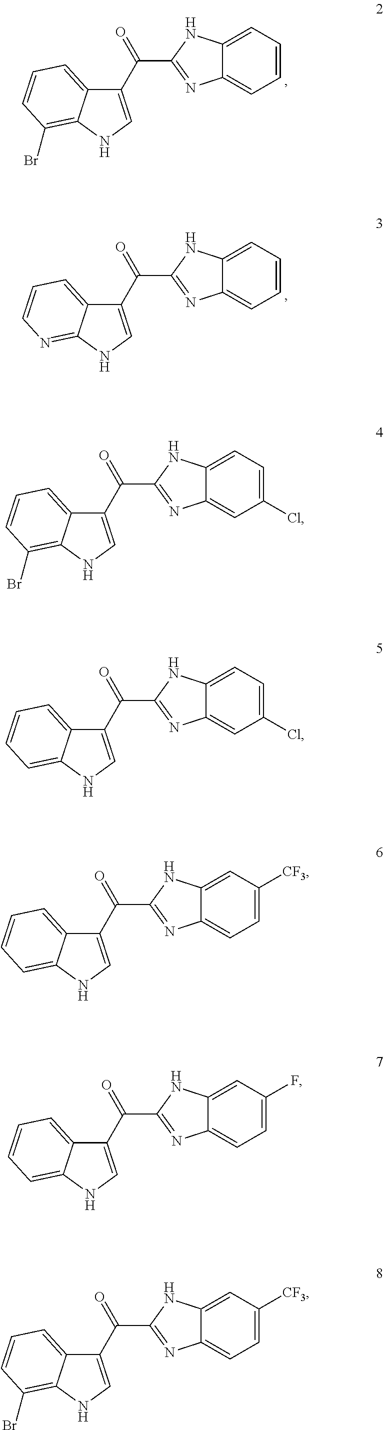

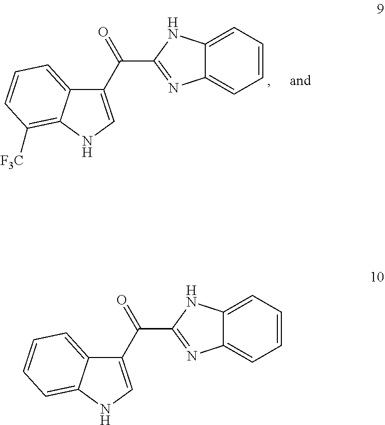 [SMILES: O=C(c1nc2ccccc2[nH]1)c1c[nH]c2c(C(F)(F)F)cccc12 ; O=C(c1nc2ccccc2[nH]1)c1c[nH]c2ccccc12]

or a pharmaceutical composition disclosed herein comprising a pharmaceutically acceptable excipient.

Some embodiments provide a method for treating a disease mediated by reactive oxygen species, inflammatory cells, Th2, Th17, Th1, IL-1, or T regulatory cell/T helper cell imbalance, or any combination thereof; the method comprising administering to a subject in need thereof, a compound having the structure of the formula (I) or (II).

Some embodiments provide a method for treating a disease mediated by reactive oxygen species, which method comprises administering to a subject an effective amount of one or more compounds of Formula (I) or (II) or a pharmaceutical composition disclosed herein comprising a pharmaceutically acceptable excipient.

Some embodiments provide a method for treating a disease mediated by inflammatory cells, which method comprises administering to a subject an effective amount of one or more compounds of Formula (I) or (II) or a pharmaceutical composition disclosed herein comprising a pharmaceutically acceptable excipient.

Some embodiments provide a method for treating a disease mediated by T cells, which method comprises administering to a subject an effective amount of one or more compounds of Formula (I) or (II) or a pharmaceutical composition disclosed herein comprising a pharmaceutically acceptable excipient.

Some embodiments provide a method for treating a disease mediated by CD3+ cells, which method comprises administering to a subject an effective amount of one or more compounds of Formula (I) or (II) or a pharmaceutical composition disclosed herein comprising a pharmaceutically acceptable excipient.

Some embodiments provide a method for treating a disease mediated by CD4+ cells, which method comprises administering to a subject an effective amount of one or more compounds of Formula (I) or (II) or a pharmaceutical composition disclosed herein comprising a pharmaceutically acceptable excipient.

Some embodiments provide a method for treating a disease mediated by CD8+ cells, which method comprises administering to a subject an effective amount of one or more compounds of Formula (I) or (II) or a pharmaceutical composition disclosed herein comprising a pharmaceutically acceptable excipient.

Some embodiments provide a method for treating a disease mediated by macrophages, which method comprises administering to a subject an effective amount of one or more compounds of Formula (I) or (II) or a pharmaceutical composition disclosed herein comprising a pharmaceutically acceptable excipient.

Some embodiments provide a method for treating a disease mediated by Th2, which method comprises administering to a subject an effective amount of one or more compounds of Formula (I) or (II) or a pharmaceutical composition disclosed herein comprising a pharmaceutically acceptable excipient. In some embodiments, the disease mediated by Th2 is associated with increased expression of IL-4. In some embodiments, the disease mediated by Th2 is associated with increased expression of IL-13. In some embodiments, the disease mediated by Th2 is associated with increased expression of IL-31. In some embodiments, the disease mediated by Th2 is associated with increased expression of thymic stromal lymphopoietin. In some embodiments, the disease mediated by Th2 is associated with increased expression IL-5. In some embodiments, the disease mediated by Th2 is associated with increased expression of IL-33.

Some embodiments provide a method for treating a disease mediated by Th17, which method comprises administering to a subject an effective amount of one or more compounds of Formula (I) or (II) or a pharmaceutical composition disclosed herein comprising a pharmaceutically acceptable excipient. In some embodiments, the disease mediated by Th17 is associated with increased expression of IL-17. In some embodiments, the disease mediated by Th17 is associated with increased expression of IL-22. In some embodiments, the disease mediated by Th17 is associated with increased expression of IL-23. In some embodiments, the disease mediated by Th17 is associated with increased expression of IL-12. In some embodiments, the disease mediated by Th17 is associated with increased expression IL-24. In some embodiments, the disease mediated by Th17 is associated with increased expression of IL-26. In some embodiments, the disease mediated by Th17 is associated with increased expression of IL-36. In some embodiments, the disease mediated by Th17 is associated with increased expression of CCL20. In some embodiments, the disease mediated by Th17 is associated with increased expression of cathelicidin. In some embodiments, the disease mediated by Th17 is associated with increased expression of defensins.

Some embodiments provide a method for treating a disease mediated by Th1, which method comprises administering to a subject an effective amount of one or more compounds of Formula (I) or (II) or a pharmaceutical composition disclosed herein comprising a pharmaceutically acceptable excipient. In some embodiments, the disease mediated by Th1 is associated with increased expression of interferons.

Some embodiments provide a method for treating a disease mediated by IL-1, which method comprises administering to a subject an effective amount of one or more compounds of Formula (I) or (II) or a pharmaceutical composition disclosed herein comprising a pharmaceutically acceptable excipient. In some embodiments, the disease mediated by IL-1 is associated with increased expression of IL-1. In some embodiments, the disease mediated by IL-1 is associated with increased expression of IL-18. In some embodiments, the disease mediated by IL-1 is associated with increased expression of IL-36. In some embodiments, the disease mediated by IL-1 is associated with increased expression of IL-37. In some embodiments, the disease mediated by IL-1 is associated with increased expression of IL-38.

Some embodiments provide a method for treating a disease mediated by T regulatory cell/T helper cell imbalance, which method comprises administering to a subject an effective amount of one or more compounds of Formula (I) or (II) or a pharmaceutical composition disclosed herein comprising a pharmaceutically acceptable excipient. In some embodiments, the T regulatory cell/T helper cell imbalance comprises T regulatory cells selected from CD4+Foxp3+ cells. In some embodiments, the T regulatory cell/T helper cell imbalance comprises T helper cells selected from CD4+ cells.

Some embodiments provide a method for treating any of the diseases disclosed herein, which method comprises a subject that is a mammal.

Some embodiments provide a method for treating any of the diseases disclosed herein, which method comprises a subject that is a human.

Some embodiments provide a method for treating any of the diseases disclosed herein, which method comprises a route of administration selected from the group consisting of enteral, intravenous, oral, intraarticular, intramuscular, subcutaneous, intraperitoneal, epidural, transdermal, transmucosal, and any combination thereof. In some embodiments, the route of administration is oral.

Further embodiments include administering a combination of compounds to the subject in need thereof. A combination can include a compound, composition, pharmaceutical composition described herein with an additional medicament.

Some embodiments include co-administering a compound, composition, and/or pharmaceutical composition described herein, with an additional medicament. By “co-administration,” it is meant that the two or more agents have a biological effect on a subject at the same time, regardless of when or how they are actually administered. In one embodiment, the agents are administered simultaneously. In one such embodiment, administration in combination is accomplished by combining the agents in a single dosage form. In another embodiment, the agents are administered sequentially. In one embodiment the agents are administered through the same route, such as orally. In another embodiment, the agents are administered through different routes, such as one being administered orally and another being administered intravenous.

In some embodiments, the compounds and compositions comprising the compounds described herein can be used to treat a host of conditions arising from a dermatologic disease, a respiratory disease, an oncologic disease, an autoimmune disease, or an immunologic disease.

To further illustrate this invention, the following examples are included. The examples should not, of course, be construed as specifically limiting the invention. Variations of these examples within the scope of the claims are within the purview of one skilled in the art and are considered to fall within the scope of the invention as described, and claimed herein. The reader will recognize that the skilled artisan, armed with the present disclosure, and skill in the art is able to prepare and use the invention without exhaustive examples. The following examples will further describe the present invention, and are used for the purposes of illustration only, and should not be considered as limiting.

### EXAMPLES

### General Procedures

It will be apparent to the skilled artisan that methods for preparing precursors and functionality related to the compounds claimed herein are generally described in the literature. In these reactions, it is also possible to make use of variants which are themselves known to those of ordinary skill in this art, but are not mentioned in greater detail. The skilled artisan given the literature and this disclosure is well equipped to prepare any of the compounds.

It is recognized that the skilled artisan in the art of organic chemistry can readily carry out manipulations without further direction, that is, it is well within the scope and practice of the skilled artisan to carry out these manipulations. These include reduction of carbonyl compounds to their corresponding alcohols, oxidations, acylations, aromatic substitutions, both electrophilic and nucleophilic, etherifications, esterification and saponification and the like. These manipulations are discussed in standard texts such as March Advanced Organic Chemistry (Wiley), Carey and Sundberg, Advanced Organic Chemistry (incorporated herein by reference in their entirety) and the like. All the intermediate compounds of the present invention were used without further purification unless otherwise specified.

The skilled artisan will readily appreciate that certain reactions are best carried out when other functionality is masked or protected in the molecule, thus avoiding any undesirable side reactions and/or increasing the yield of the reaction. Often the skilled artisan utilizes protecting groups to accomplish such increased yields or to avoid the undesired reactions. These reactions are found in the literature and are also well within the scope of the skilled artisan. Examples of many of these manipulations can be found for example in T. Greene and P. Wuts Protecting Groups in Organic Synthesis, 4th Ed., John Wiley & Sons (2007), incorporated herein by reference in its entirety.

The following example schemes are provided for the guidance of the reader, and represent preferred methods for making the compounds exemplified herein. These methods are not limiting, and it will be apparent that other routes may be employed to prepare these compounds. Such methods specifically include solid phase based chemistries, including combinatorial chemistry. The skilled artisan is thoroughly equipped to prepare these compounds by those methods given the literature and this disclosure. The compound numberings used in the synthetic schemes depicted below are meant for those specific schemes only, and should not be construed as or confused with same numberings in other sections of the application.

Trademarks used herein are examples only and reflect illustrative materials used at the time of the invention. The skilled artisan will recognize that variations in lot, manufacturing processes, and the like, are expected. Hence the examples, and the trademarks used in them are non-limiting, and they are not intended to be limiting, but are merely an illustration of how a skilled artisan may choose to perform one or more of the embodiments of the invention.

The following abbreviations have the indicated meanings:
DCM=dichloromethane
DMSO=dimethyl sulfoxide
eq=equivalent
HRMS=high resolution mass spectrometry
ITE=2-(1′H-indole-3′-carbonyl)-thiazole-4-carboxylic acid methyl ester
NMR=nuclear magnetic resonance
rt=room temperature

The following example schemes are provided for the guidance of the reader, and collectively represent an example method for making the compounds provided herein. Furthermore, other methods for preparing compounds described herein will be readily apparent to the person of ordinary skill in the art in light of the following reaction schemes and examples. Unless otherwise indicated, all variables are as defined above.

### Example 1

### Preparation of Compound 10

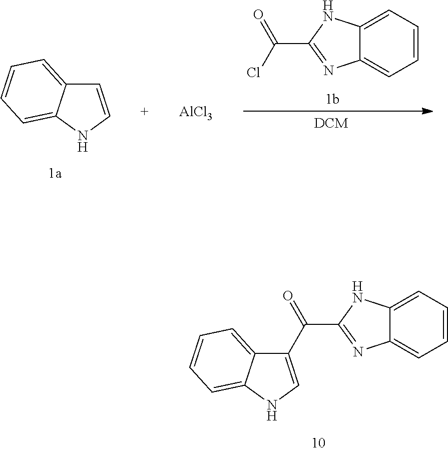 [SMILES: c1ccc2[nH]ccc2c1 ; "AlCl3" ; O=C(Cl)c1nc2ccccc2[nH]1 ; O=C(c1nc2ccccc2[nH]1)c1c[nH]c2ccccc12]

Indole 1a (1 eq) was added to a round bottom flask equipped with a magnetic stir bar, followed by dichloromethane (DCM) and aluminum chloride (AlC₃, 1-3 eq) under a stream of nitrogen (N₂). The solution was stirred at rt for 1 hour, resulting in a colorless solution. Acid chloride 1b (1 eq) dissolved in DCM was added dropwise to the flask under N₂. The reaction was allowed to stir overnight at rt under N₂. The magnetic stir bar was removed and celite was added for dry loading and purification using a Combiflash Rf system. The product was eluted with an CH₃CN:H₂O gradient with 0.1% formic acid as a modifier, followed by an additional round of silica purification. Compound 10 was obtained as a white solid. ¹H NMR (400 MHz, CDCl₃); MS (HRMS) m/z [M+H]⁺262.0982, ([M+H]⁺ calcd. for C₁₆H₁₂N₃O, 262.0980).

### Preparation of Compound 2

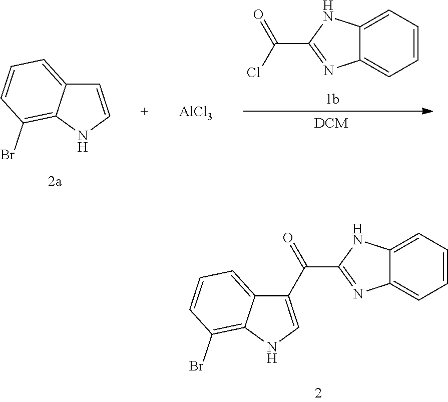 [SMILES: Brc1cccc2cc[nH]c12 ; "AlCl3" ; O=C(Cl)c1nc2ccccc2[nH]1 ; O=C(c1nc2ccccc2[nH]1)c1c[nH]c2c(Br)cccc12]

Compound 2 was prepared by following the procedure described for compound 1 using indole 2a (1 eq) in place of indole 1a. Compound 2 (1H-benzo[d]imidazol-2-yl) (7-bromo-1H-indol-3-yl)methanone was obtained as a white solid. ¹H NMR (400 MHz, CDCl₃); MS (HRMS) m/z [M+H]⁺ 340.0109, ([M+H]⁺ calcd. for C₁₆H₁₁BrN₃O, 340.0085)

### Preparation of Compound 3

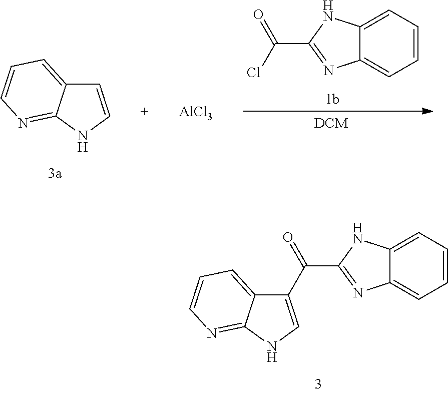 [SMILES: c1cnc2[nH]ccc2c1 ; "AlCl3" ; O=C(Cl)c1nc2ccccc2[nH]1 ; O=C(c1nc2ccccc2[nH]1)c1c[nH]c2ncccc12]

Compound 2 was prepared by following the procedure described for compound 1 using indole 2a (1 eq) in place of indole 1a. Compound 3 (1H-benzo[d]imidazol-2-yl) (1H-pyrrolo[2,3-b]pyridin-3-yl)methanone was obtained as a white solid. MS (HRMS) m/z [M+H]⁺ 263.099, ([M+H]⁺ calcd for C₁₅H₁₁N₄O, 263.0933).

### Preparation of Compound 4

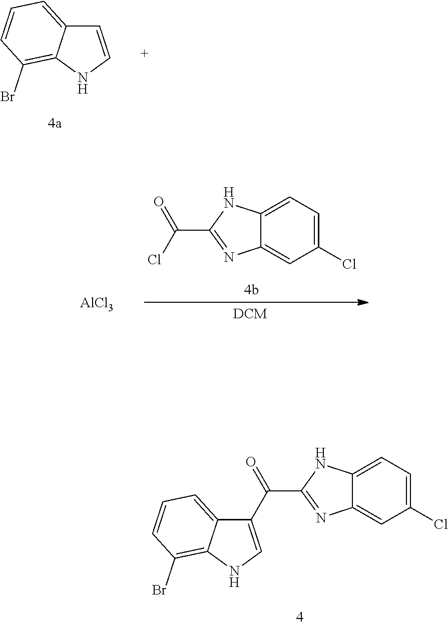 [SMILES: Brc1cccc2cc[nH]c12 ; O=C(Cl)c1nc2cc(Cl)ccc2[nH]1 ; "AlCl3" ; O=C(c1nc2cc(Cl)ccc2[nH]1)c1c[nH]c2c(Br)cccc12]

Compound 4 was prepared by following the procedure described for compound 1 using indole 4a (1 eq) in place of indole 1a and acid chloride 4b (1 eq) in place of acid chloride 1b. Compound 4 (7-bromo-1H-indol-3-yl) (5-chloro-1H-benzo[d]imidazol-2-yl)methanone was obtained as a white solid. MS (HRMS) m/z 375.96924 [M+2+H]⁺, 373.97084 [M+H]⁺ (calcd for C₁₆H₁₀BrClN₃O, 373.9696).

### Preparation of Compound 5

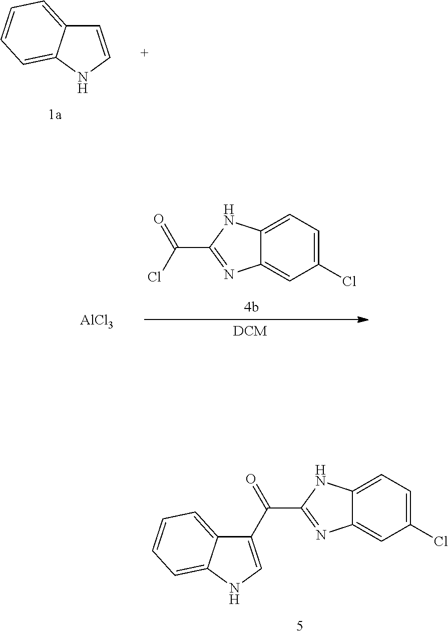 [SMILES: c1ccc2[nH]ccc2c1 ; O=C(Cl)c1nc2cc(Cl)ccc2[nH]1 ; "AlCl3" ; O=C(c1nc2cc(Cl)ccc2[nH]1)c1c[nH]c2ccccc12]

Compound 5 was prepared by following the procedure described for compound 1 using acid chloride 4b (1 eq) in place of acid chloride 1b. Compound 5 (5-chloro-1H-benzo[d]imidazol-2-yl) (1H-indol-3-yl)methanone was obtained as a white solid. MS (HRMS) m/z [M+H]⁺ 296.06306, ([M+H]⁺ calcd for C₁₆H₁₁ClN₃O, 296.0595).

### Preparation of Compound 6

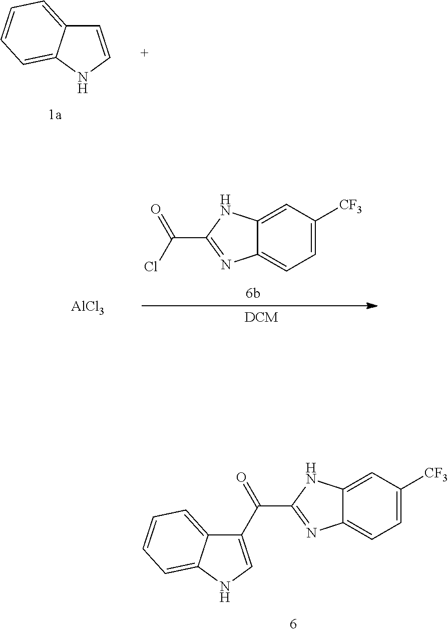 [SMILES: c1ccc2[nH]ccc2c1 ; O=C(Cl)c1nc2ccc(C(F)(F)F)cc2[nH]1 ; "AlCl3" ; O=C(c1nc2ccc(C(F)(F)F)cc2[nH]1)c1c[nH]c2ccccc12]

Compound 6 is prepared by following the procedure described for compound 1 using acid chloride 6b (1 eq) in place of acid chloride 1b. Compound 6 (1H-indol-3-yl) (6-(trifluoromethyl)-1H-benzo[d]imidazol-2-yl)methanone is obtained as a white solid.

### Preparation of Compound 7

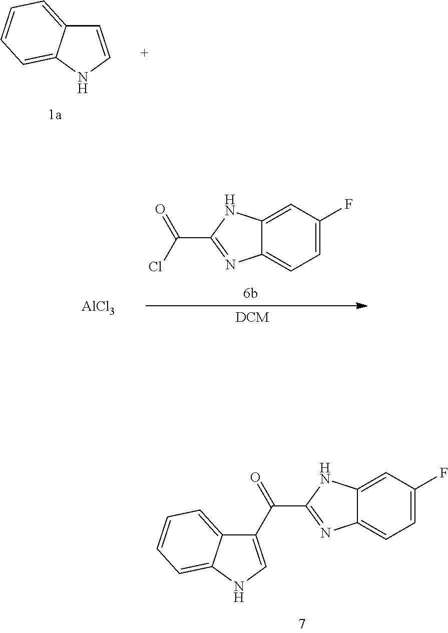 [SMILES: c1ccc2[nH]ccc2c1 ; O=C(Cl)c1nc2ccc(F)cc2[nH]1 ; "AlCl3" ; O=C(c1nc2ccc(F)cc2[nH]1)c1c[nH]c2ccccc12]

Compound 7 is prepared by following the procedure described for compound 1 using acid chloride 7b (1 eq) in place of acid chloride 1b. Compound 7 (6-fluoro-1H-benzo[d]imidazol-2-yl) (1H-indol-3-yl)methanone is obtained as a white solid.

### Preparation of Compound 8

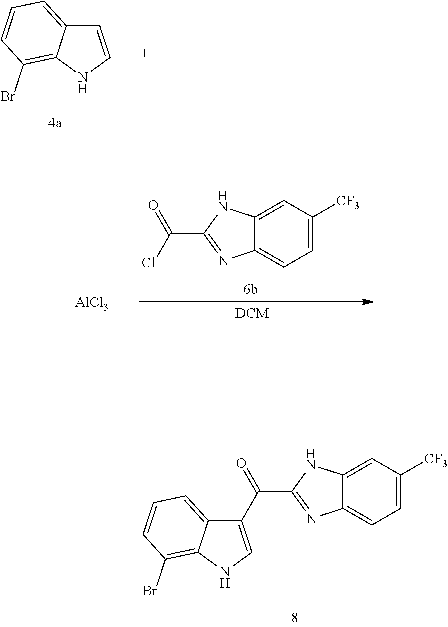 [SMILES: Brc1cccc2cc[nH]c12 ; O=C(Cl)c1nc2ccc(C(F)(F)F)cc2[nH]1 ; "AlCl3" ; O=C(c1nc2ccc(C(F)(F)F)cc2[nH]1)c1c[nH]c2c(Br)cccc12]

Compound 8 is prepared by following the procedure described for compound 1 using indole 4a (1 eq) in place of indole 1a and acid chloride 6b (1 eq) in place of acid chloride 1b. Compound 8 (7-bromo-1H-indol-3-yl) (6-(trifluoromethyl)-1H-benzo[d]imidazol-2-yl)methanone is obtained as a white solid.

### Preparation of Compound 9

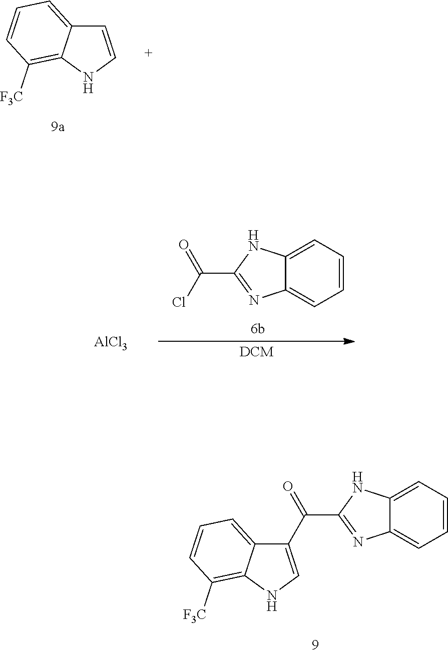 [SMILES: FC(F)(F)c1cccc2cc[nH]c12 ; O=C(Cl)c1nc2ccccc2[nH]1 ; "AlCl3" ; O=C(c1nc2ccccc2[nH]1)c1c[nH]c2c(C(F)(F)F)cccc12]

Compound 9 is prepared by following the procedure described for compound 1 using indole 9a (1 eq) in place of indole 1a. Compound 9 ((1H-benzo[d]imidazol-2-yl) (7-(trifluoromethyl)-1H-indol-3-yl)methanone is obtained as a white solid.

### Example 2

### Gene Expression Biomarker Activity Screen

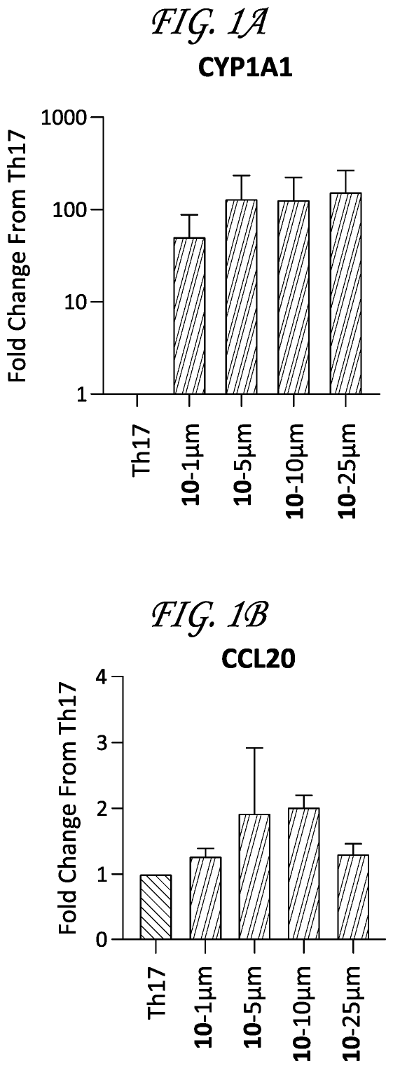

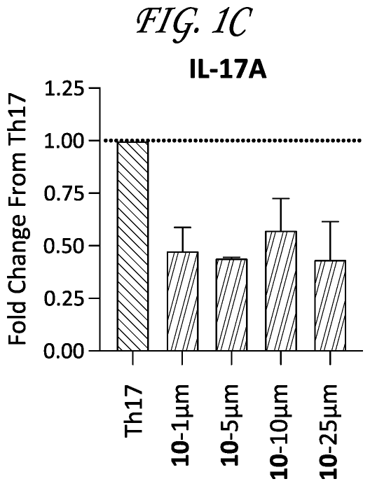

Compound 10 and comparative compound tapinarof are evaluated for their ability to inhibit production of gene expression biomarkers using the biomarkers panel described in the below tables. Each compound is dissolved in DMSO at a dilution volume of 1000× and the resultant solutions are stored at −80° C. Concentrations tested include 1 μM, 5 μM, 10 μM, and 25 μM. Freshly excised healthy human skin is dermatomed to an approximate thickness of 750±100 μm using an Integra® dermatome according to US-SOP-3005 and US-SOP-3006. The dermatomed skin is sectioned into 7 mm biopsies and placed into 6.5 mm permeable membrane Transwell® inserts with the Stratum corneum side apical and a section of collagen between the basal dermal tissue and the permeable membrane (n=4 per test sample). The Transwell® inserts have an average surface area of approximately 0.33 cm² and a volume of 0.5 mL. The basal chambers of the inserts are filled with 0.5 mL of Cornification media and the compound solutions are added to the media to a concentration of 10 μM. The inserts are stored in a humidified incubator overnight at 37° C. for approximately 16 hours. The contents of the basal chambers are vacuum aspirated and replaced with 0.5 mL of pre-warmed (ca. 37° C.) Cornification media containing the compound at a concentration of 10 μM and stimulation cocktail. Tissue explants are harvested at 24 hours post stimulation to measure production of the gene expression biomarkers by quantitative reverse transcription polymerase chain reaction (RT-qPCR). Results of inhibition of the production of the gene expression biomarkers are reported as a percentage of detected cytokine compared to the stimulated control and a fold change in gene expression compared to the untreated control. The test samples include Compound 10, untreated vehicle (control), TH1 stimulation control, TH2 stimulation control, TH17 stimulation control, tapinarof. The results of the inhibition of production of CYP1A1, CCL20, and IL-17A by Compound 10 are shown in FIGS. 1A, 1B, and 1C, respectively.

### Th1 Stimulation Panel

| Gene | | | |
|---|---|---|---|
| acronym | Gene name | Primary cell types | Activity |
| Cox 2 | Cyclooxygenase 2 | ubiquitous | Prostaglandin |
| (PTGS2) | | | production |
| IFNγ | Interferon gamma | T-lymphocyte | Activates macrophages |
| IL5 | interleukin 5 | Th2 cells | Eosinophil activation |
| IL22 | Interleukin-22 | T lymphocyte | Pro-inflammatory |
| IL6 | interleukin 6 | T cells, macrophages | Pro-inflammatory |
| CXCL10 | C-X-C motif | Monocytes, | IFNγ responsive, |
| | chemokine 10 | fibroblast | immune cell |
| | | | chemoattractant |
| IL1β | Interleukin 1 beta | Macrophages | Pro-inflammatory |
| GM-CSF | Granulocyte-macrophage | Macrophage, | Monocyte maturation |
| | colony-stimulating | T cell, | |
| | factor | fibroblasts | |
| IL2 | Interleukin-2 | Lymphocytes | T cell activation |
| IL4 | interleukin 4 | Lymphocytes | Th2 differentiation |
| CLDN23 | Claudin 23 | Keratinocytes | Tight junction |
| S100A12 | S100 calcium-binding | Keratinocytes | Pro-inflammatory |
| | protein A12 | | |
| S100A9 | S100 calcium-binding | Keratinocytes | Lymphocyte |
| | protein A9 | | recruitment |
| DefB4 | Defensin Beta 4 | Keratinocyte | Antimicrobial |
| TNFα | Tumor necrosis factor α | Keratinocyte | Pro-inflammatory |
| IL12 | Interleukin 12 | Dendritic cells, | Th1 cell |
| | | macrophage | differentiation |

### Th2 Stimulation Panel

| Gene | | | |
|---|---|---|---|
| acronym | Gene name | Primary cell types | Activity |
| IL13 | Interleukin 13 | Th2 cells | Pro-Inflammatory |
| IL31 | Interleukin-31 | Th2 cells | Pro-inflammatory |
| IL5 | interleukin 5 | Th2 cells | Eosinophil activation |
| IL22 | Interleukin-22 | T lymphocyte | Pro-inflammatory |
| GM-CSF | Granulocyte-macrophage | Macrophage, | Monocyte maturation |
| | colony-stimulating | T cell, | |
| | factor | fibroblasts | |
| CXCL1 | chemokine (C-X-C motif) | Macrophage, | Neutrophil recruitment |
| | ligand 1 | neutrophil | |
| MMP12 | Matrix metallopeptidase | Macrophage, | STAT responsive, |
| | 12 | dendritic | elastase, ECM |
| | | cell | remodelling |
| IL2 | Interleukin-2 | Lymphocytes | T cell activation |
| IL4 | interleukin 4 | Lymphocytes | Th2 differentiation |
| CCL26 | C-C motif chemokine | Keratinocyte | Eosinophil recruitment |
| (Eotaxin 3) | 26 | | |
| Def84 | Defensin Beta 4 | Keratinocyte | Antimicrobial |
| Fil | Filaggrin | Keratinocyte | Barrier function |
| TSLP | Thymic stromal | Fibroblasts, | Immune cell |
| | lymphopoietin | keratinocytes | activation |
| CCL17(TARC) | C-C motif chemokine | Dendritic cells | T cell recruitment |
| | 17 | | |

### Th17 Stimulation Panel

| Gene acronym | Gene name | Primary cell types | Activity |
|---|---|---|---|
| IL17α | Interleukin 17a | T-lymphocyte | Pro-inflammatory |
| IL17f | Interleukin 17f | T-lymphocyte | Pro-inflammatory |
| IL22 | Interleukin 22 | T-lymphocyte | Pro-inflammatory |
| IFNγ | Interferon gamma | T-lymphocyte | Activates macrophages |
| IL19 | Interleukin 19 | Monocytes, | Anti-inflammatory |
| | | B-lymphocyte | |
| CCL4 | C-C motif chemokine | Macrophage, | Chemokine for NK |
| | ligand 4 | T-lymphocyte | cells, monocytes |
| IL23p19 | Interleukin 23, p19 | Macrophage, | Activates MMP9 |
| | | dendritic cell | |
| CCL20 | C-C motif chemokine | Macrophage, | Chemokine for |
| | ligand 20 | keratinocyte | lymphocytes |
| CXCL10 | chemokine (C-X-C | Monocyte, | IFNγ responsive |
| | motif) ligand 10 | fibroblast | |
| CXCL1 | chemokine (C-X-C | Fibroblast, | Chemokine for |
| | motif) ligand 1 | keratinocyte | neutrophils |
| IL6 | Interleukin 6 | Fibroblast, | Pro-inflammatory, |
| | | keratinocyte | anti-inflammatory |
| TNFα | Tumor necrosis | Keratinocyte | Pro-inflammatory |
| | factor α | | |
| S100A7 | Psoriasin | Keratinocyte | Antimicrobial |
| S100A12 | Calgranulin C | Keratinocyte | Antimicrobial |
| DefB4 | Defensin Beta 4 | Keratinocyte | Antimicrobial |
| SerpinB4 | Serpin family 8 | Keratinocyte | Serine protease |
| | member 4 | | inhibitor |
| CXCL6 | chemokine (C-X-C | Keratinocyte | Chemokine for |
| | motif) ligand 6 | | neutrophils |
| IL36G | Interleukin 36 G | Keratinocyte | Pro-inflammatory |
| IL1β | Interleukin 1β | Keratinocyte, macrophage | Pro-inflammatory |
| IL8 (CXCL8) | chemokine (C-X-C | Keratinocyte, macrophage | Chemokine for |
| | motif) ligand 8 | | neutrophils |

### Example 3

### Imiquimod (IMQ)-Induced Skin Inflammation

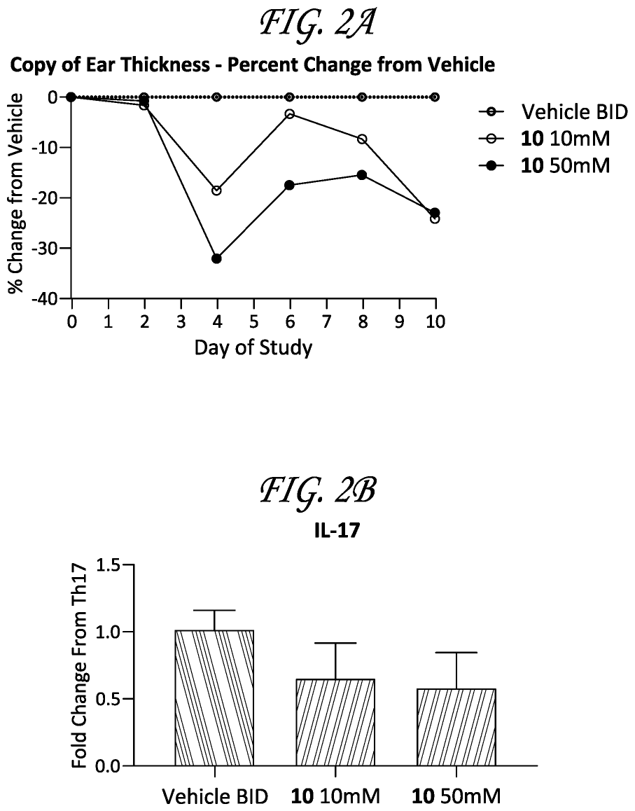

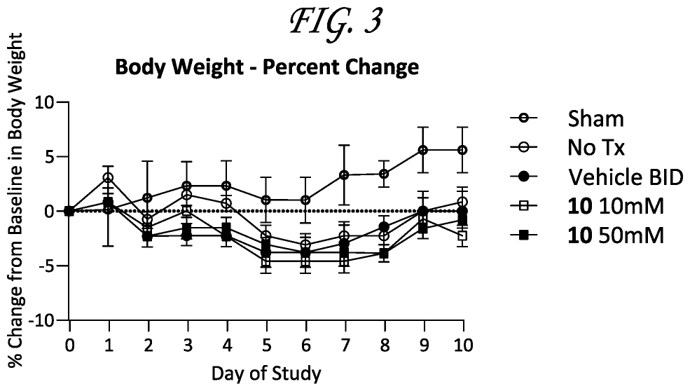

Compound 10, comparative compound tapinarof, clobetasol (positive control), and the vehicle (DMSO) are administered topically to groups of six Male Balb/c mice weighing 22±2 g. The compounds are administered on the right ear once daily from Day 1 to Day 9 for 9 consecutive days. Compound 10 and tapinarof daily dosage is 20 μL, 50 mM. Daily Clobetasol dosage is 20 mg of 0.05% cream. One hour after administration, 15 mg imiquimod (IMQ) cream (5%, Aldaras) is applied topically on the right ear from Day 1 to Day 9 for 9 consecutive days. Ear swelling is measured (mm) as an index of inflammation on Day 0 and thereafter 30 min before dosing on Days 2, 4, 6 and 8. On Day 10, ear swelling is measured 24 hours after the last dosing. Ear edema is calculated by subtracting the thickness of Day 0 (normal control) from Day n (treated ear). Percent inhibition is calculated according to the formula: (Lc−Lt)/Lc×100, where Lc and Lt refer to increase of ear thickness in control and treated mice, respectively. Clinical scoring is done on the same days as the measurement of ear swelling. Body weight is recorded on Day 0 and thereafter daily before dosing. On Day 10, animals are sacrificed, blood is collected from all mice and serum processed and kept at −20° C. for optional analysis. Right ears are harvested, weighed after the last ear swelling measurement, and cut into two parts, one part to be kept in 10% formalin for histopathological examination and the second part to be stored in RNAlater® solution at −20° C. for detection of IL-17 A, IL-22, GAPDH, and beta defensin with quantitative real-time PCR (qPCR). Dermal thickness is measured after taking photographs under a light microscope for the histopathological examination sections. The dermal thickness is defined as the mean distance between the epidermal-dermal junction and the dermal-subcutaneous fat junction measured at 5 different skin sections of each mouse. The results of change in ear thickness and the inhibition of production of IL-17 in the car by Compound 10 are shown in FIGS. 2A and 2B, respectively. The results of change in body weight induced by Compound 10 is shown in FIG. 3. The results indicate that Compound 10 resulted in significant reductions in ear swelling compared to vehicle and reduction in IL-17 gene expression, whereas there was no significant effect on body weights.

### Example 4

### Psoriasis Human Tissue Model

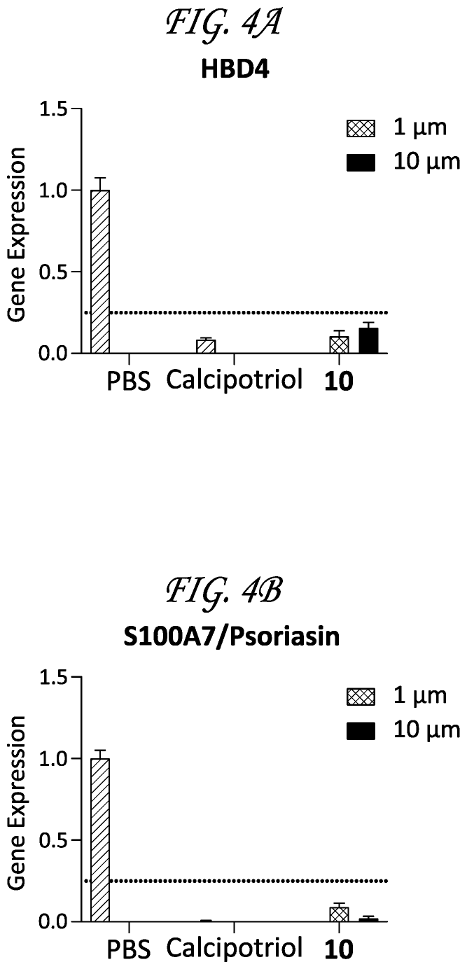

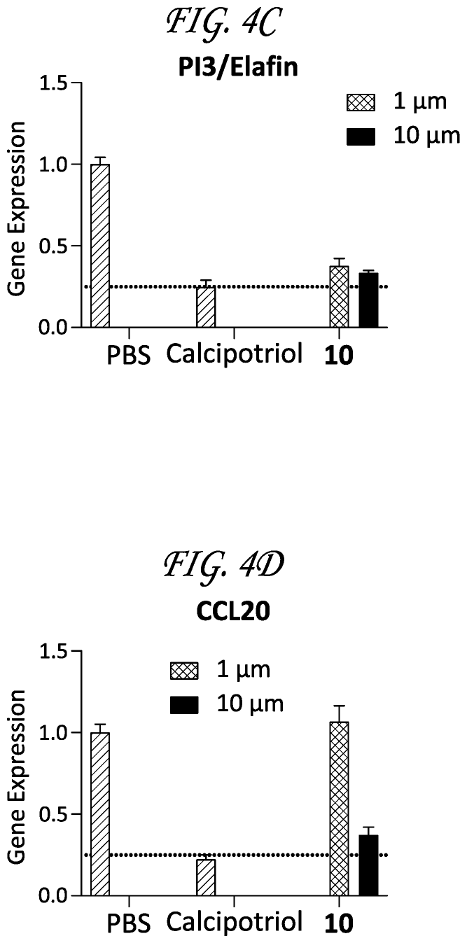

Compound 10, comparative compound tapinarof, calcipotriol positive control (PC), and PBS negative control (NC) are applied topically and basolaterally to tissue groups (n=3) fed with 5 ml SOR-300-MM media. Compound 10 and tapinarof dosage is 1 μM topically and 10 μM basolaterally. PC dosage is 2.5 μM/mL in media. NC dosage is 50 μL. After treatment the tissues are incubated for 96 hours at 37±1° C. and 5±1% CO₂. At 48 hours post-treatment, the tissues are rinsed with Cₐ⁺⁺Mg⁺⁺ Free-Dulbecco's PBS (DPBS) three times×2 seconds. The tissues are re-dosed and incubated for an additional 48 hours as described herein. RNA is isolated to monitor gene expression levels of human beta-defensin (HBD4), Psoriasin (S100A7), Elafin (PI3 peptidase inhibitor 3), and C—C motif chemokine ligand 20 (CCL-20) by qPCR analysis. The results of the inhibition of production of HBD4, S100A7, Elafin, and CCL20 by Compound 10 are shown in FIGS. 4A, 4B, 4C, and 4D, respectively. A 75% inhibition of production is indicted by a dotted line in FIGS. 4A, 4B, and 4C.

### Example 5

### Inflammation in Human Keratinocytes and T-Cells

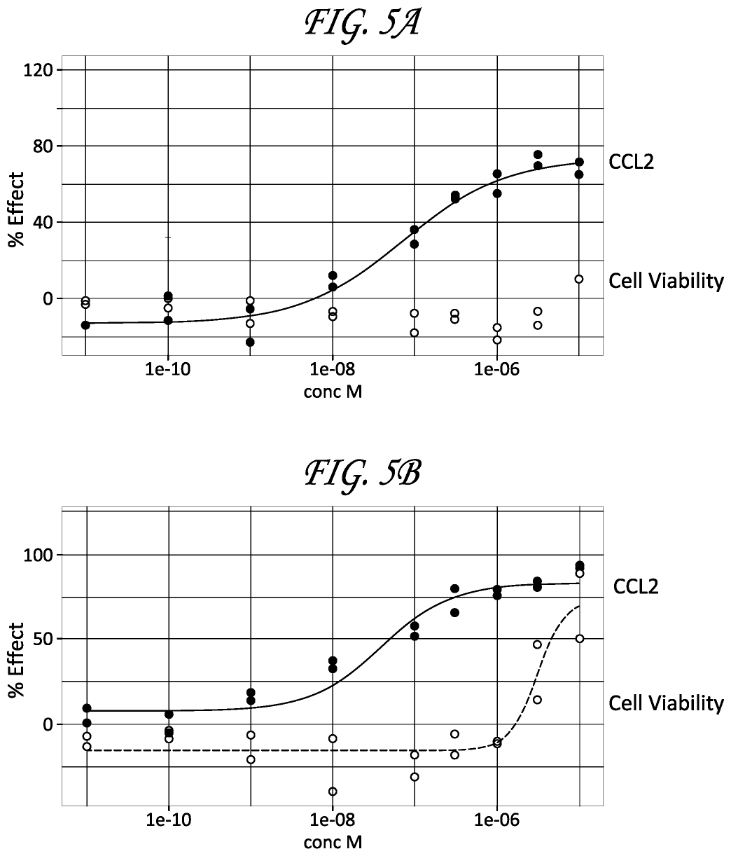

Inflammation of human keratinocytes was induced by IL-4, IL-13, IL-22 and IFN-γ. The cells were exposed to Compound 10 or tapiranof and measured for inhibition of CCL2 release and cell viability. FIG. 5A depicts concentration response curves of CCL2 release and cell viability for Compound 10. FIG. 5B depicts concentration response curves of CCL2 release and cell viability for tapiranof. Both Compound 10 and tapiranof inhibited release of CCL2. However, tapiranof resulted in inhibition of cell viability at higher concentrations, whereas Compound 10 did not affect cell viability.

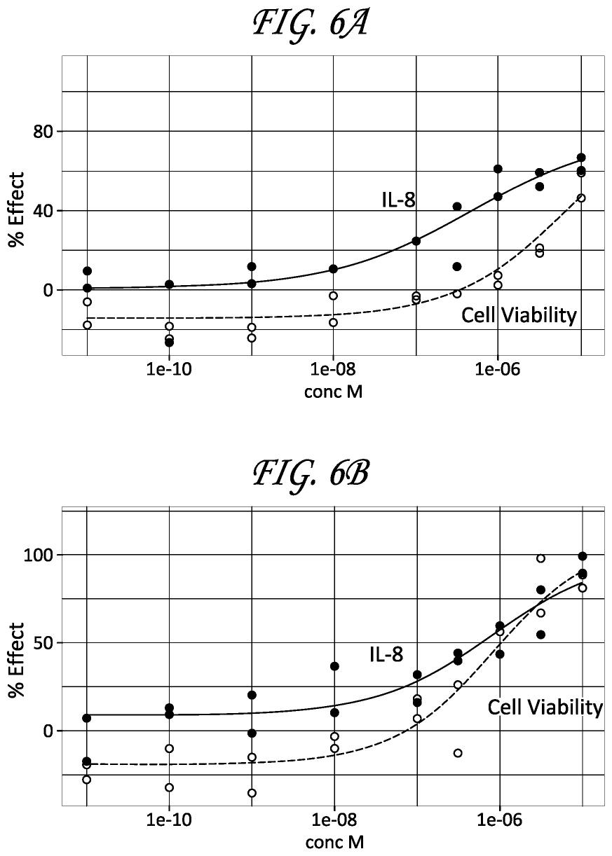

Inflammation of human keratinocytes was also induced by IL-17 and IFN-α. The cells were exposed to Compound 10 or tapiranof and measured for inhibition of IL-8 release and cell viability. FIG. 6A depicts concentration response curves of inhibition of IL-8 release and cell viability for Compound 10. FIG. 6B depicts concentration response curves of inhibition of IL-8 release and cell viability for tapiranof. Both Compound 10 and tapiranof inhibited release of IL-8 and had similar inhibition of viability.

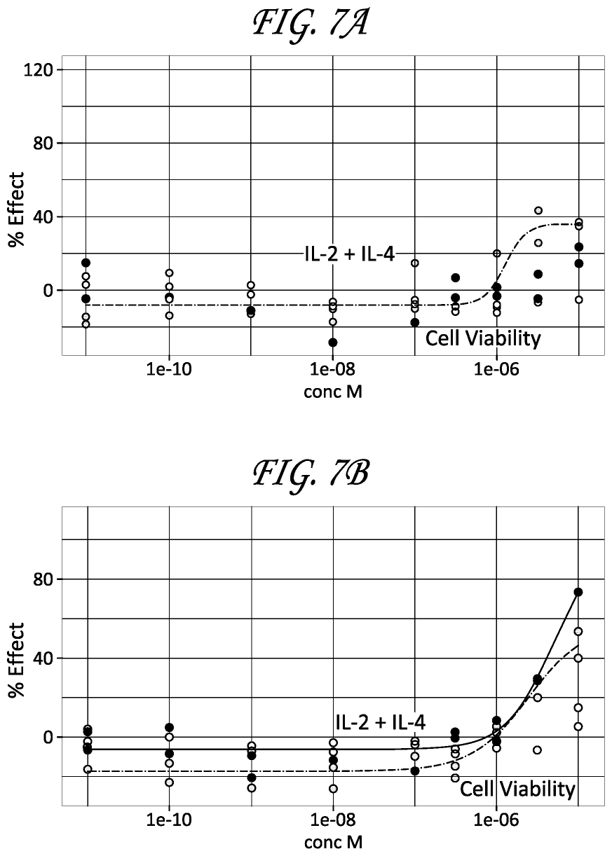

Inflammation of T-cells were induced by CD2, CD3, and CD28. The cells were exposed to Compound 10 or tapiranof and measured for inhibition of IL-2 and IL-4 release and cell viability. FIG. 7A depicts concentration response curves of inhibition of IL-2 and IL-4 release and cell viability for Compound 10. FIG. 7B depicts concentration response curves of inhibition of IL-2 and IL-4 release and cell viability for tapiranof. Both Compound 10 and tapiranof inhibited release of IL-2 and IL-4 at the highest test concentrations.

While some embodiments have been illustrated and described, a person with ordinary skill in the art, after reading the foregoing specification, can effect changes, substitutions of equivalents and other types of alterations to the compounds of the present technology or salts, pharmaceutical compositions, derivatives, prodrugs, metabolites, tautomers or racemic mixtures thereof as set forth herein. Each aspect and embodiment described above can also have included or incorporated therewith such variations or aspects as disclosed in regard to any or all of the other aspects and embodiments.

The present technology is also not to be limited in terms of the particular aspects described herein, which are intended as single illustrations of individual aspects of the present technology. Many modifications and variations of this present technology can be made without departing from its spirit and scope, as will be apparent to those skilled in the art. Functionally equivalent methods within the scope of the present technology, in addition to those enumerated herein, will be apparent to those skilled in the art from the foregoing descriptions. Such modifications and variations are intended to fall within the scope of the appended claims. It is to be understood that this present technology is not limited to particular methods, reagents, compounds, compositions, labeled compounds or biological systems, which can, of course, vary. It is also to be understood that the terminology used herein is for the purpose of describing particular aspects only, and is not intended to be limiting. Thus, it is intended that the specification be considered as exemplary only with the breadth, scope and spirit of the present technology indicated only by the appended claims, definitions therein and any equivalents thereof.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

In addition, where features or aspects of the disclosure are described in terms of Markush groups, those skilled in the art will recognize that the disclosure is also thereby described in terms of any individual member or subgroup of members of the Markush group. Each of the narrower species and subgeneric groupings falling within the generic disclosure also form part of the present technology. This includes the generic description of the present technology with a proviso or negative limitation removing any subject matter from the genus, regardless of whether or not the excised material is specifically recited herein.

All publications, patent applications, issued patents, and other documents (for example, journals, articles and/or textbooks) referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims, along with the full scope of equivalents to which such claims are entitled.

What is claimed is:

1. A compound having the structure of the formula I: 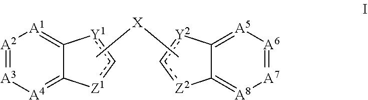
or a pharmaceutically acceptable salt thereof, wherein:
A¹, A², A³, A⁴, A⁵, A⁶, A⁷, and A⁸ are each independently C(R¹) or N, wherein at least one of A¹, A², A³, A⁴, A⁵, A⁶, A⁷, and A⁸ is C(R⁶) or N;
Y¹, Y¹, Z¹, and Z² are each independently selected from the group consisting of O, S, C(R²), and NR³, wherein at least one of Y¹, Y², Z¹, and Z² is NR³;
X is selected from the group consisting of (C(R⁴)₂)ₘ, (C═NR⁵), (C═O), (C═S), O, S, and SO₂;
each R¹ is independently selected from the group consisting of hydrogen, halo, hydroxy, optionally substituted C₁₋₈ alkyl, optionally substituted C₁₋₈ haloalkyl, optionally substituted C₁₋₈ alkoxy, optionally substituted C₆₋₁₀ aryloxy, optionally substituted C₁₋₈ alkyl-O-sulfonyl, optionally substituted C₁₋₈ haloalkyl-O-sulfonyl, optionally substituted C₃₋₁₀ carbocyclyl, optionally substituted C₄₋₁₀ carbocyclyl(C₁-C₆)alkyl, optionally substituted 3-10 membered heterocyclyl, optionally substituted 3-10 membered heterocyclyl(C₁-C₆)alkyl, optionally substituted C₆₋₁₀ aryl, optionally substituted C₆₋₁₀ aryl(C₁-C₆)alkyl, optionally substituted 5-10 membered heteroaryl, and optionally substituted 5-10 membered heteroaryl(C₁-C₆)alkyl, or independently two R¹ groups can be taken together with the atoms to which they are attached to form a ring selected from the group consisting of optionally substituted 5-10 membered heterocyclic, optionally substituted 5-10 membered heteroaryl, optionally substituted C₆₋₁₀ aryl, and optionally substituted C₄₋₁₀ carbocyclyl;
each R² and R³ is independently selected from the group consisting of hydrogen, halo, hydroxy, and optionally substituted C₁₋₈alkyl or is absent;
each R⁴ is each independently selected from the group consisting of hydrogen, halo, hydroxy, and optionally substituted C₁₋₈alkyl;
R⁵ is hydrogen or C₁₋₈ alkyl;
each R⁶ is independently selected from the group consisting of hydroxy, bromo, chloro, optionally substituted C₁₋₈ haloalkyl, optionally substituted C₆₋₁₀ aryloxy, optionally substituted C₁₋₈ haloalkyl-O-sulfonyl, optionally substituted C₃₋₁₀ carbocyclyl, optionally substituted C₄₋₁₀ carbocyclyl(C₁-C₆)alkyl, optionally substituted 3-10 membered heterocyclyl, optionally substituted 3-10 membered heterocyclyl(C₁-C₆)alkyl, optionally substituted C₆₋₁₀ aryl, optionally substituted C₆₋₁₀ aryl(C₁-C₆)alkyl, optionally substituted 5-10 membered heteroaryl, and optionally substituted 5-10 membered heteroaryl(C₁-C₆)alkyl, or independently two R⁶ groups can be taken together with the atoms to which they are attached to form a ring selected from the group consisting of optionally substituted 5-10 membered heterocyclic, optionally substituted 5-10 membered heteroaryl, optionally substituted C₆₋₁₀ aryl, and optionally substituted C₄₋₁₀ carbocyclyl;
m is an integer from 1 to 6; and
each [symbol] is independently single bond or double bond, wherein adjacent [symbol] are not both double bonds.

2. The compound of claim 1, wherein A¹ is N.

3. The compound of claim 1 or claim 2, wherein A² is N.

4. The compound of any one of claims 1 to 3, wherein A³ is N.

5. The compound of any one of claims 1 to 4, wherein A⁴ is N.

6. The compound of any one of claims 1 to 5, wherein A⁵ is N.

7. The compound of any one of claims 1 to 6, wherein A⁶ is N.

8. The compound of any one of claims 1 to 7, wherein A⁷ is N.

9. The compound of any one of claims 1 to 8, wherein A⁸ is N.

10. The compound of any one of claims 1 to 9, wherein at least two of A¹, A², A³, A⁴, A⁵, A⁶, A⁷, and A⁸ are N.

11. The compound of any one of claims 1 to 10, wherein at least three of A¹, A², A³, A⁴, A⁵, A⁶, A⁷, and A⁸ are N.

12. The compound of any one of claims 1 to 11, wherein at least four of A¹, A², A³, A⁴, A⁵, A⁶, A⁷, and A⁸ are N.

13. The compound of any one of claims 1 and 3 to 12, wherein A¹ is C(R¹).

14. The compound of any one of claims 1, 2 and 4 to 13, wherein A² is C(R¹).

15. The compound of any one of claims 1 to 3 and 5 to 14, wherein A³ is C(R¹).

16. The compound of any one of claims 1 to 4 and 6 to 15, wherein A⁴ is C(R¹).

17. The compound of any one of claims 1 to 5 and 7 to 16, wherein A⁵ is C(R¹).

18. The compound of any one of claims 1 to 6 and 8 to 17, wherein A⁶ is C(R¹).

19. The compound of any one of claims 1 to 7 and 9 to 18, wherein A⁷ is C(R¹).

20. The compound of any one of claims 1 to 8 and 10 to 19, wherein A⁸ is C(R¹).

21. The compound of any one of claims 1, 3 to 12, and 14 to 20, wherein A¹ is C(R⁶).

22. The compound of any one of claims 1, 2, 4 to 13, and 15 to 21, wherein A² is C(R⁶).

23. The compound of any one of claims 1 to 3, 5 to 14, and 16 to 22, wherein A³ is C(R⁶).

24. The compound of any one of claims 1 to 4, 6 to 15, and 17 to 23, wherein A⁴ is C(R⁶).

25. The compound of any one of claims 1 to 5, 7 to 16, and 18 to 24, wherein A⁵ is C(R⁶).

26. The compound of any one of claims 1 to 6, 8 to 17, and 19 to 25, wherein A⁶ is C(R⁶).

27. The compound of any one of claims 1 to 7, 9 to 18, and 20 to 26, wherein A⁷ is C(R⁶).

28. The compound of any one of claims 1 to 8, 10 to 19, and 21 to 27, wherein A⁸ is C(R⁶).

29. The compound of any one of claims 1 to 28, wherein at least one R¹ is hydrogen.

30. The compound of any one of claims 1 to 28, wherein at least one R¹ is halo.

31. The compound of any one of claims 1 to 28, wherein at least one R¹ is F.

32. The compound of any one of claims 1 to 28, wherein at least one R¹ is CL.

33. The compound of any one of claims 1 to 28, wherein at least one R¹ is Br.

34. The compound of any one of claims 1 to 28, wherein at least one R¹ is Cr-8 alkyl.

35. The compound of any one of claims 1 to 28, wherein at least one R¹ is methyl.

36. The compound of any one of claims 1 to 28, wherein at least one R¹ is C₁₋₈ haloalkyl.

37. The compound of any one of claims 1 to 28, wherein at least one R¹ is CF₃.

38. The compound of any one of claims 1 to 37, wherein at least one R⁶ is C₁₋₈ haloalkyl.

39. The compound of any one of claims 1 to 37, wherein at least one R⁶ is CF₃.

40. The compound of any one of claims 1 to 39, wherein Y¹ is C(R²).

41. The compound of any one of claims 1 to 40, wherein Z¹ is C(R²).

42. The compound of any one of claims 1 to 41, wherein Y² is C(R²).

43. The compound of any one of claims 1 to 42, wherein Z² is C(R²).

44. The compound of any one of claims 1 to 39 and 41 to 43, wherein Y¹ is NR³.

45. The compound of any one of claims 1 to 40 and 42 to 44, wherein Z¹ is NR³.

46. The compound of any one of claims 1 to 41 and 43 to 45, wherein Y² is NR³.

47. The compound of any one of claims 1 to 42 and 44 to 46, wherein Z² is NR³.

48. The compound of any one of claims 1 to 39, wherein Y¹ is C(R²) and Z¹ is NR³.

49. The compound of claim 48, wherein Y² and Z² are C(R²).

50. The compound of claim 48, wherein Y² is C(R²) and Z² is NR³.

51. The compound of claim 48, wherein Y² is NR³ and Z² is C(R²).

52. The compound of claim 48, wherein Y² and Z² are NR³.

53. The compound of any one of claims 1 to 52, wherein at least one R² is hydrogen.

54. The compound of any one of claims 1 to 52, wherein at least one R² is C₁₋₈ alkyl.

55. The compound of any one of claims 1 to 52, wherein at least one R² is absent.

56. The compound of any one of claims 1 to 55, wherein at least one R³ is hydrogen.

57. The compound of any one of claims 1 to 55, wherein at least one R³ is C₁₋₈ alkyl.

58. The compound of any one of claims 1 to 55, wherein at least one R³ is absent.

59. The compound of any one of claims 1 to 58, wherein X is (C(R⁴)₂)ₘ.

60. The compound of claim 59, wherein at least one R⁴ is hydrogen.

61. The compound of any one of claims 59 to 60, wherein m is 1.

62. The compound of any one of claims 1 to 58, wherein X is (C═NR⁵).

63. The compound of any one of claims 1 to 62, wherein R⁵ is hydrogen.

64. The compound of any one of claims 1 to 58, wherein X is (C═O).

65. The compound of any one of claims 1 to 58, wherein X is (C═S).

66. The compound of any one of claims 1 to 58, wherein X is O.

67. The compound of any one of claims 1 to 58, wherein X is SO₂.

68. The compound of claim 1 having the structure of the formula Ia: 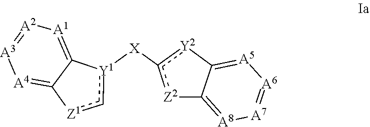
or a pharmaceutically acceptable salt thereof wherein:
A¹, A², A³, A⁴, A⁵, A⁶, A⁷, and A⁸ are each independently C(R¹) or N, wherein at least one of A¹, A², A³, A⁴, A⁵, A⁶, A⁷, and A⁸ is C(R⁶) or N; and
Y¹, Y², Z¹, and Z² are each independently selected from the group consisting of O, S, C(R²), and NR³, wherein at least one of Y¹, Y², Z¹, and Z² is NR³.

69. The compound of claim 68, wherein Y¹ is C(R²) and Z¹ is NR³.

70. The compound of claim 69, wherein Y² and Z¹ are C(R²).

71. The compound of claim 69, wherein Y² is C(R²) and Z² is NR³.

72. The compound of claim 69, wherein Y² is NR³ and Z² is C(R²).

73. The compound of claim 69, wherein Y² and Z² are NR³.

74. The compound of any one of claims 68 to 73, wherein R² is hydrogen.

75. The compound of any one of claims 68 to 73, wherein R² is absent.

76. The compound of any one of claims 68 to 75, wherein R¹ is hydrogen.

77. The compound of any one of claims 68 to 75, wherein R³ is absent.

78. The compound of any one of claims 68 to 77, wherein X is (C(R⁴)₂)ₘ.

79. The compound of claim 78, wherein at least one R⁴ is hydrogen.

80. The compound of any one of claims 78 to 79, wherein m is 1.

81. The compound of any one of claims 68 to 77, wherein X is (C═O)).

82. The compound of claim 1 having the structure of the formula Ia-2: 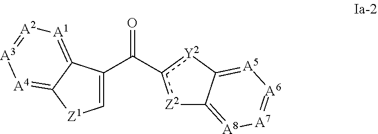
or a pharmaceutically acceptable salt thereof.

83. The compound of claim 82, wherein Z¹ is NR³.

84. The compound of claim 83, wherein Y² is C(R²) and Z² is NR³.

85. The compound of claim 83, wherein Y² is NR³ and Z¹ is C(R²).

86. The compound of claim 83, wherein Y² and Z² are NR³.

87. The compound of any one of claims 82 to 86, wherein at least one R³ is hydrogen.

88. The compound of any one of claims 82 to 86, wherein at least one R³ is absent.

89. The compound of claim 1 having the structure of the formula Ia-3: 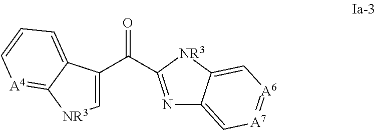
or a pharmaceutically acceptable salt thereof.

90. The compound of claim 89, wherein Ad is C(R¹).

91. The compound of any one of claims 89 to 90, wherein A⁶ is C(R¹).

92. The compound of any one of claims 89 to 91, wherein A⁷ is C(R¹).

93. The compound of any one of claims 89 to 92, wherein at least one R¹ is hydrogen.

94. The compound of any one of claims 89 to 92, wherein at least one R¹ is halo.

95. The compound of any one of claims 89 to 92, wherein at least one R¹ is F.

96. The compound of any one of claims 89 to 92, wherein at least one R¹ is Cl.

97. The compound of any one of claims 89 to 92, wherein at least one R¹ is Br.

98. The compound of any one of claims 89 to 92, wherein at least one R¹ is Cr-z alkyl.

99. The compound of any one of claims 89 to 92, wherein at least one R¹ is methyl.

100. The compound of any one of claims 89 to 92, wherein at least one R¹ is C₁₋₈ haloalkyl.

101. The compound of any one of claims 89 to 92, wherein at least one R¹ is CF₃.

102. The compound of any one of claims 89 to 101, wherein at least one R³ is hydrogen.

103. The compound of any one of claims 89 to 101, wherein at least one R³ is C₁₋₈ alkyl.

104. The compound of any one of claims 89 to 101, wherein at least one R³ is absent.

105. The compound of any one of claims 89 to 104, wherein at least one R⁶ is C₁₋₈ haloalkyl.

106. The compound of any one of claims 89 to 104, wherein at least one R⁶ is CF₃.

107. The compound of any one of claim 1, having the structure selected from: 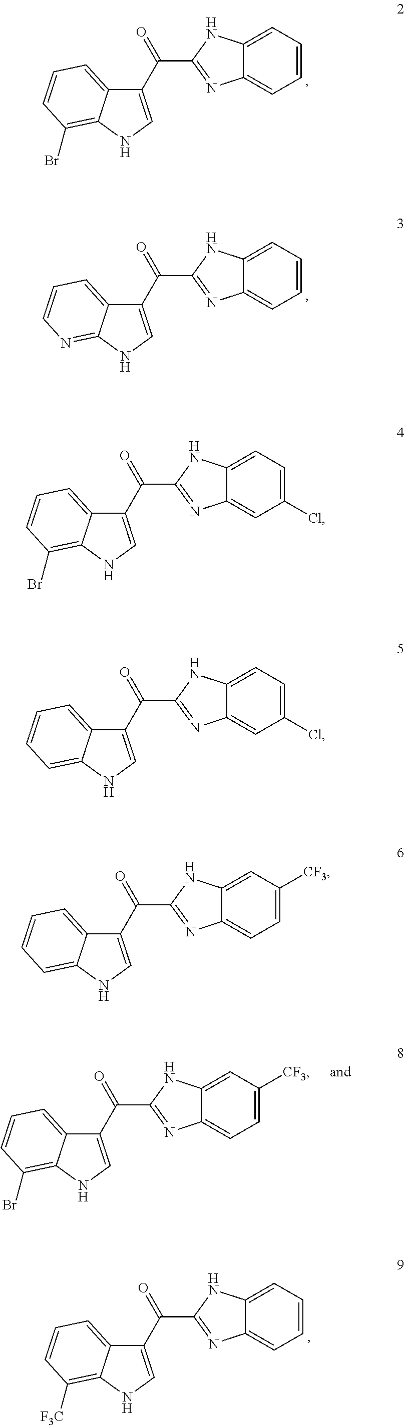
or a pharmaceutically acceptable salt thereof.

108. A compound having the structure: 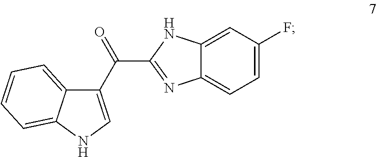 [SMILES: O=C(c1nc2ccc(F)cc2[nH]1)c1c[nH]c2ccccc12]
or a pharmaceutically acceptable salt thereof.

109. A pharmaceutical composition comprising a therapeutically effective amount of a compound of any one of claims 1 to 108 and a pharmaceutically acceptable excipient.

110. A method of treating a disease, wherein the disease is a dermatologic, respiratory, oncologic, autoimmune, or immunologic disease, the method comprising administering to a subject in need thereof, the compound of any one of claims 1 to 108.

111. The method of claim 110, wherein the disease is a dermatologic disease.

112. The method of claim 111, wherein the disease is atopic dermatitis, eczema, psoriasis, contact dermatitis, vitiligo, hidradenitis suppurativa, lichen planus, ichthyosis, palmoplantar pustulosis, dermatomyositis, netherton's syndrome, fibrosis, scleroderma, basal cell carcinoma, squamous cell carcinoma, merkel cell carcinoma, skin aging, bullous pemphigoid, sarcoidosis, chronic urticaria, rosacea, alopecia areata, pyoderma gangrenosum, cutaneous T cell lymphoma, or any combination thereof.

113. The method of claim 110, wherein the disease is a respiratory disease.

114. The method of claim 113, wherein the disease is asthma, chronic obstructive pulmonary disorder, idiopathic pulmonary fibrosis, bronchitis, cystic fibrosis, acute respiratory distress syndrome, interstitial lung disease, or any combination thereof.

115. The method of claim 110, wherein the disease is an oncologic disease.

116. The method of claim 115, wherein the disease is melanoma, non-small cell lung cancer, prostate cancer, pancreatic cancer, bladder cancer, mesothelioma, or any combination thereof.

117. The method of claim 110, wherein the disease is an autoimmune disease.

118. The method of claim 117, wherein the disease is ankylosing spondylitis, rheumatoid arthritis, psoriatic arthritis, systemic lupus erythematosus, diabetic nephropathy, Graves' disease, multiple sclerosis, type 1 diabetes, Sjogren's disease, neuromyelitis optica, amyloidosis, IgG4 disease, Behcet's disease, Celiac disease, fibromyalgia, ulcerative colitis, Crohn's disease, uveitis, or any combination thereof.

119. The method of claim 110, wherein the disease is an immunologic disease.

120. The method of claim 119, wherein the disease is COVID-19, SARS-COV2, graft versus host disease, cytokine release syndrome, STAT3 gain of function, interferonopathies, Kawasaki disease, multi-inflammatory syndrome, or any combination thereof.

121. The method of any one of claims 110 to 120, wherein the method comprises administering the pharmaceutical composition of claim 109.

122. A method of treating a disease, wherein the disease is a dermatologic disease selected from hidradenitis suppurativa, lichen planus, ichthyosis, palmoplantar pustulosis, dermatomyositis, netherton's syndrome, fibrosis, scleroderma, bullous pemphigoid, chronic urticaria, rosacea, alopecia areata, pyoderma gangrenosum, or any combination thereof, a respiratory disease selected from idiopathic pulmonary fibrosis, cystic fibrosis, acute respiratory distress syndrome, interstitial lung disease, or any combination thereof, an autoimmune disease selected from diabetic nephropathy, Graves' disease, neuromyelitis optica, IgG4 disease, Celiac disease, fibromyalgia, uveitis, or any combination thereof, or an immunologic disease selected from COVID-19, SARS-COV2, cytokine release syndrome, STAT3 gain of function, interferonopathies, multi-inflammatory syndrome, or any combination thereof, the method comprising administering to a subject in need thereof, a compound having the structure of the formula II: 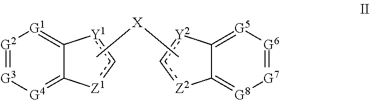
or a pharmaceutically acceptable salt thereof, wherein:
G¹, G², G³, G⁴, G⁵, G⁶, G⁷, and GR are each independently C(R¹) or N;
Y¹, Y², Z¹, and Z² are each independently selected from the group consisting of O, S, C(R²), and NR³ and at least one of Y¹, Y², Z¹, and Z² is NR³;
X is selected from the group consisting of (C(R⁴)₂)ₘ, (C═NR⁵), (C═O), (C═S), O, S, and SO₂;
each R¹ is independently selected from the group consisting of hydrogen, halo, hydroxy, optionally substituted C₁₋₈ alkyl, optionally substituted C₁₋₈ haloalkyl, optionally substituted Cis alkoxy, optionally substituted C₆₋₁₀ aryloxy, optionally substituted C₁₋₈ alkyl-O-sulfonyl, optionally substituted C₁₋₈ haloalkyl-O-sulfonyl, optionally substituted C₃₋₁₀ carbocyclyl, optionally substituted C₄₋₁₀ carbocyclyl(C₁-C₆)alkyl, optionally substituted 3-10 membered heterocyclyl, optionally substituted 3-10 membered heterocyclyl(C₁-C₆)alkyl, optionally substituted C₆₋₁₀ aryl, optionally substituted C₆₋₁₀ aryl(C₁-C₆)alkyl, optionally substituted 5-10 membered heteroaryl, and optionally substituted 5-10 membered heteroaryl(C₁-C₆)alkyl, or independently two R¹ groups can be taken together with the atoms to which they are attached to form a ring selected from the group consisting of optionally substituted 5-10 membered heterocyclic, optionally substituted 5-10 membered heteroaryl, optionally substituted C₆₋₁₀ aryl, and optionally substituted C₄₋₁₀ carbocyclyl;
each R² and R³ is independently selected from the group consisting of hydrogen, halo, hydroxy, and optionally substituted C₁₋₈ alkyl or is absent;
each R⁵ is each independently selected from the group consisting of hydrogen, halo, hydroxy, and optionally substituted C₁₋₈ alkyl;
R⁵ is hydrogen or C₁₋₈ alkyl;
m is an integer from 1 to 6; and
each [symbol] is independently single bond or double bond, wherein adjacent [symbol] are not both double bonds.

123. The method of claim 122, wherein the disease is hidradenitis suppurativa, lichen planus, ichthyosis, palmoplantar pustulosis, dermatomyositis, netherton's syndrome, fibrosis, scleroderma, bullous pemphigoid, chronic urticaria, rosacea, alopecia areata, pyoderma gangrenosum, or any combination thereof.

124. The method of claim 122, wherein the disease is idiopathic pulmonary fibrosis, cystic fibrosis, acute respiratory distress syndrome, interstitial lung disease, or any combination thereof.

125. The method of claim 122, wherein the disease is diabetic nephropathy, Graves' disease, neuromyelitis optica, IgG4 disease, Celiac disease, fibromyalgia, uveitis, or any combination thereof.

126. The method of claim 122, wherein the disease is COVID-19, SARS-COV2, cytokine release syndrome, STAT3 gain of function, interferonopathies, multi-inflammatory syndrome, or any combination thereof.

127. The method of claim 122, wherein the compound is selected from: 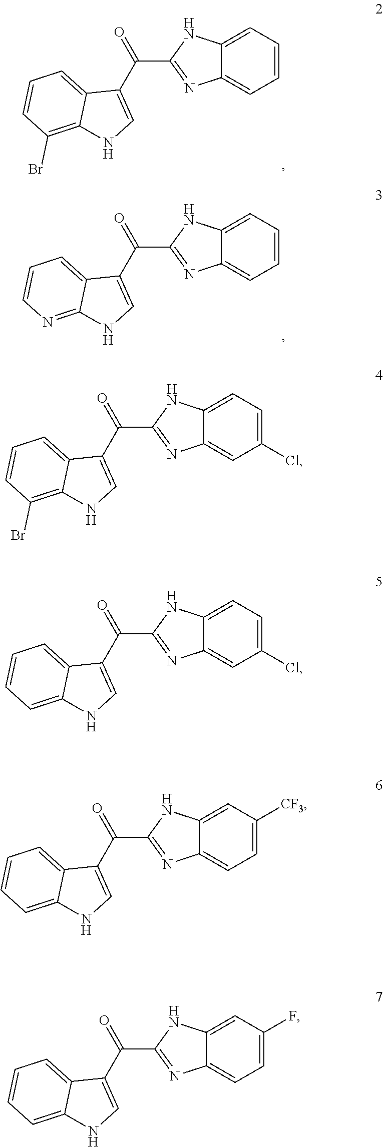 [SMILES: O=C(c1nc2ccccc2[nH]1)c1c[nH]c2c(Br)cccc12 ; O=C(c1nc2ccccc2[nH]1)c1c[nH]c2ncccc12 ; O=C(c1nc2cc(Cl)ccc2[nH]1)c1c[nH]c2c(Br)cccc12 ; O=C(c1nc2cc(Cl)ccc2[nH]1)c1c[nH]c2ccccc12 ; O=C(c1nc2ccc(C(F)(F)F)cc2[nH]1)c1c[nH]c2ccccc12 ; O=C(c1nc2ccc(F)cc2[nH]1)c1c[nH]c2ccccc12] 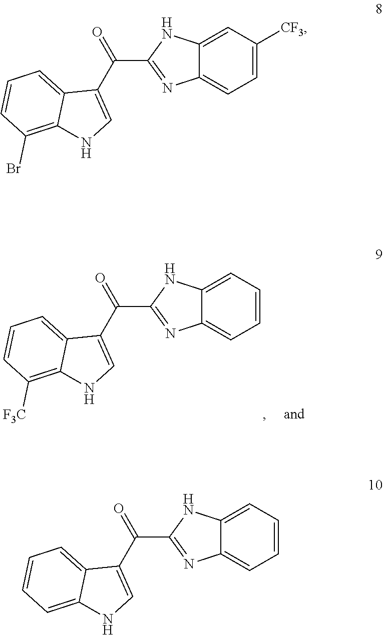 [SMILES: O=C(c1nc2ccc(C(F)(F)F)cc2[nH]1)c1c[nH]c2c(Br)cccc12 ; O=C(c1nc2ccccc2[nH]1)c1c[nH]c2c(C(F)(F)F)cccc12 ; O=C(c1nc2ccccc2[nH]1)c1c[nH]c2ccccc12]
or a pharmaceutically acceptable salt thereof.

128. A method of treating a disease mediated by reactive oxygen species, inflammatory cells, Th2, Th17, Th1, IL-1, or T regulatory cell/T helper cell imbalance, comprising administering to a subject in need thereof, a compound having the structure of the formula II: 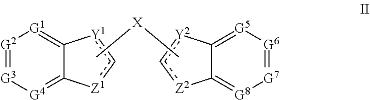
or a pharmaceutically acceptable salt thereof, wherein:
G¹, G², G³, G⁴, G⁵, G⁶, G⁷, and G⁸ are each independently C(R¹) or N;
Y¹, Y², Z¹, and Z² are each independently selected from the group consisting of O, S, C(R²), and NR³ and at least one of Y¹, Y², Z¹, and Z² is NR³;
X is selected from the group consisting of (C(R⁴)₂)ₘ, (C═NR⁵), (C═O), (C═S), O, S, and SO₂;
each R¹ is independently selected from the group consisting of hydrogen, halo, hydroxy, optionally substituted Cis alkyl, optionally substituted C₁₋₈ haloalkyl, optionally substituted C₁₋₈ alkoxy, optionally substituted C₆₋₁₀ aryloxy, optionally substituted C₁₋₈ alkyl-O-sulfonyl, optionally substituted Cis haloalkyl-O-sulfonyl, optionally substituted C₃₋₁₀ carbocyclyl, optionally substituted C₄₋₁₀ carbocyclyl(C₁-C₆)alkyl, optionally substituted 3-10 membered heterocyclyl, optionally substituted 3-10 membered heterocyclyl(C₁-C₆)alkyl, optionally substituted C₆₋₁₀ aryl, optionally substituted C₆₋₁₀ aryl(C₁-C₆)alkyl, optionally substituted 5-10 membered heteroaryl, and optionally substituted 5-10 membered heteroaryl(C₁-C₆)alkyl, or independently two R¹ groups can be taken together with the atoms to which they are attached to form a ring selected from the group consisting of optionally substituted 5-10 membered heterocyclic, optionally substituted 5-10 membered heteroaryl, optionally substituted C₆₋₁₀ aryl, and optionally substituted C₄₋₁₀ carbocyclyl;
each R² and R³ is independently selected from the group consisting of hydrogen, halo, hydroxy, and optionally substituted Cis alkyl or is absent;
each R⁴ is each independently selected from the group consisting of hydrogen, halo, hydroxy, and optionally substituted C₁₋₈ alkyl;
R⁵ is hydrogen or C₁₋₈ alkyl;
m is an integer from 1 to 6; and
each [symbol] is independently single bond or double bond, wherein adjacent [symbol] are not both double bonds.

129. The method of claim 128, wherein the disease is mediated by aryl hydrocarbon receptor.

130. The method of claim 128, wherein the inflammatory cells are selected from T cells, macrophages, and any combination thereof.

131. The method of claim 130, wherein the T cells are selected from CD3+ cells, CD4+ cells, CD8+ cells, and any combination thereof.

132. The method of claim 128, wherein the disease is mediated by Th2.

133. The method of claim 132, wherein the disease is associated with increased expression of one or more of IL-4, IL-13, IL-31, thymic stromal lymphopoietin, IL-5, or IL-33.

134. The method of claim 128, wherein the disease is mediated by Th17.

135. The method of claim 134, wherein the disease is associated with increased expression of one or more IL-17, IL-22, IL-23, IL-12, IL-24, IL-26, IL-36, CCL20, cathelicidin, or defensins.

136. The method of claim 128, wherein the disease is mediated by Th1.

137. The method of claim 136, wherein the disease is associated with increased expression of interferons.

138. The method of claim 128, wherein the disease is mediated by IL-1.

139. The method of claim 138, wherein the disease is associated with increased expression of one or more IL-1, IL-18, IL-36, IL-37, or IL-38.

140. The method of claim 128, wherein the disease is mediated by T regulatory cell/T helper cell imbalance.

141. The method of claim 140, wherein the T regulatory cell/T helper cell imbalance comprises T regulatory cells selected from CD4+Foxp3+ cells.

142. The method of claim 140 or claim 141, wherein the T regulatory cell/T helper cell imbalance comprises T helper cells selected from CD4+ cells.

143. The method of any one of claims 110 to 142, wherein the subject is a mammal.

144. The method of any one of claims 110 to 143, wherein the subject is a human.

145. The method of any one of claims 110 to 144, wherein the route of administration is selected from the group consisting of enteral, intravenous, oral, intraarticular, intramuscular, subcutaneous, intraperitoneal, epidural, transdermal, transmucosal, and any combination thereof.

146. The method of any one of claims 110 to 145, wherein the administration is oral.
